(19) United States
(12) Patent Application Publication
(10) Pub. No.: US 2001/0053549 A1
(43) Pub. Date: Dec. 20, 2001

(54) Loading method

(76) Inventors: Anthony P. McHale, Portstewart (GB); Roger Craig, Smallwood (GB); Emma Jane Fadlon, Portstewart (GB)

Correspondence Address: Kathleen M. Williams, Ph.D, Palmer & Dodge, LLP, One Beacon Street, Boston, MA, 02108, US

(21) Appl. No.: 09/748,789

(22) Filed: Dec. 22, 2000

Related U.S. Application Data
(63) Continuation-in-part of application No. PCT/GB00/03056, filed on Aug. 9, 2000.
(60) Provisional application No. 60/181,796, filed on Feb. 11, 2000.

(30) Foreign Application Priority Data
Feb. 8, 2000 (GB) 0002856.3

(51) Int. Cl. C12N 15/01

(52) U.S. Cl. 435/446; 435/173.1

(57) ABSTRACT

We describe a method of producing a red blood cell suitable for delivery of an agent to a vertebrate, the method comprising: (a) providing a red blood cell; (b) pre-sensitising the red blood cell; and (c) loading the red blood cell with an agent. Use of an electric field and/or ultrasound to increase the efficiency of loading of an agent into a red blood cell is also described.

### FIELD OF THE INVENTION

The present invention relates to a method for loading a red blood cell with an agent, which cell may be sensitised to assist in agent release.

### BACKGROUND TO THE INVENTION

The delivery of therapeutic agents to specific tissues is desirable typically to ensure that a sufficiently high dose of a given agent is delivered to a selected tissue. Moreover, it is often the case that the therapeutic agent, although advantageously having beneficial therapeutic effects on the diseased tissue, may have undesirable side effects on tissues that are not diseased. For example, in the treatment of certain types of disorders, such as cancer, it is necessary to use a high enough dose of a drug to kill the cancer cells without killing an unacceptable high number of normal cells. Thus, one of the major challenges of disease treatment is to identify ways of exploiting cellular drug delivery vehicles to incorporate and to selectively release agents at a desired target site.

It has been suggested that red blood cells may be exploited as active agent/drug delivery vehicles (DeLoach & Sprandel 1985, Bibliotheca Haematologica; Publ. Karger, Munich) as it is possible to incorporate agents into human red blood cells using a variety of techniques. An example of such a technique is the exploitation of osmotic shock and modifications thereof such as hypotonic shock and subsequent recovery of isotonicity and reverse hypotonic dialysis (Luque & Pinilla, 1993, Ind. Farmac. 8, 53-59).

An alternative method for loading drugs and active agents into red blood cells is electroporation. Using this process, the agent of interest is mixed with the live red blood cells in a buffer medium and short pulses of high electric fields are applied. The red blood cell membranes are transiently made porous and the agents of interest enter the cells. The electroporation process is advantageous as very high loading indices can be achieved within a very short time period (Flynn et al., 1994, Cancer Letts., 82, 225-229).

When packaging/carrier/delivery systems such as red blood cells are used as in vivo delivery systems, they suffer from the drawback that the delivery function is dependent upon both an accumulation of the red blood cells and a breakdown of the red blood cell membrane in or at the relevant tissue/site. As a result, attempts have been made to incorporate sensitising agents into cell carriers in order to facilitate both the accumulation and/or release of an agent of interest at a target site.

By way of example, our UK Patent Applications 9816583.0 and 9826676.0 (incorporated by reference) relate inter alia to the incorporation of a dye compound, such as a porphyrin, which renders a loaded red blood cell susceptible to laser light treatment at a target site. This phenomenon, known as photodynamic activation, is exploited in order to achieve accumulation of the carrier vehicle at the relevant site and to achieve load release at that site.

Alternative energy sources have been investigated as tools for inducing payload release from loaded and sensitised cells. By way of example, ultrasound has been investigated as an alternative to light induced photodynamic activation as it has a broader degree of focus and it penetrates more deeply into the body. However, although ultrasound has also been applied to effect red blood cell lysis in vitro, its use has been limited in that its effect is only significant at lower cell concentrations (1-6×10⁶ cells) (Brayman et al., 1996, Ultrasound in Med & Biol., 22: 497-514). Moreover, ultrasound is non-specific in its effects, resulting in lysis of both loaded and endogenous red blood cells.

Recently, it has been found that certain dye compounds, in particular porphyrins, can achieve a cytopathogenic effect when the disease site is subjected to ultrasound. This technique is referred to as sonodynamic therapy and is discussed in WO98/52609. WO98/52609 teaches that ultrasound may be useful in treating disease but only when it is combined with an effective amount of an ultrasound-susceptibility modification agent such as a porphyrin.

In our U.K. patent application No. 9917416.1, incorporated by reference, we have shown that treatment of red blood cells with an electric field increases their sensitivity to ultrasound mediated disruption. Consequently, efficient unloading of therapeutic agents carried by red blood cells at a site of interest can be achieved at lower exposures of ultrasound, reducing possible damage to normal red blood cells.

### SUMMARY OF THE INVENTION

We have now further shown that if the red blood cells are pre-sensitised prior to a dialysis loading step, close to 100% efficiency in the loading step can be achieved. A first aspect of the invention relates to this finding.

However we have also found that the dialysis loading step reduces the sensitivity of the loaded cells to ultrasound. This reduction in sensitivity can be reversed by subjecting the cells to an additional sensitisation step regardless of whether the additional step is performed before or after loading. Using this pre-sensitisation/sensitisation procedure, red blood cells can be produced that have both excellent loading characteristics and ultrasound sensitivity. Consequently, highly efficient unloading of therapeutic agents carried by red blood cells at a site of interest can be achieved at low exposures of ultrasound. This represents a considerable improvement over prior art methods. The present invention therefore provides an improved method for selectively releasing an agent from a loaded red blood cell at a target site, as further aspects.

According to a first aspect of the present invention, we provide a method of producing a red blood cell suitable for delivery of an agent to a vertebrate, the method comprising: (a) providing a red blood cell; (b) pre-sensitising the red blood cell; and (c) loading the red blood cell with an agent.

Preferably, the amount of agent that is loaded into a pre-sensitised red blood cell is higher than the amount loaded into a red blood cell which is not pre-sensitised.

More preferably, the method further comprises the step of electrosensitising the cell to render it more susceptible to disruption by exposure to a stimulus, the loading step and the electrosensitisation step being performed in any order.

There is provided, according to a second aspect of the present invention, a method for selectively releasing an agent from a red blood cell comprising the steps of: (a) pre-sensitising a red blood cell; (b) loading the cell with an agent; (c) electrosensitising the cell; and (d) causing the agent to be released from the sensitised cell by applying ultrasound at a frequency and energy sufficient to cause disruption of the sensitised cell but insufficient to cause disruption of un-sensitised red blood cells, in which steps (b) and (c) can be performed in any order.

We provide, according to a third aspect of the present invention, a method for delivering an agent to a target site in a vertebrate, the method comprising a method according to the second aspect of the invention, with the further step of introducing the cell into a vertebrate between steps (c) and (d).

The red blood cell may be PEGylated prior to being introduced into the vertebrate. Preferably, the vertebrate is a mammal. In each of the above-mentioned methods, either or both of the pre-sensitising step and the electrosensitising step may be an in vitro or ex-vivo procedure.

The pre-sensitising may comprise a step of applying an electric field to the red blood cell. Alternatively, or in addition, the pre-sensitising may comprise a step of applying ultrasound to the red blood cell.

In a preferred embodiment of the invention, the red blood cell is loaded with the agent by hypotonic dialysis.

Preferably, the electrosensitising comprises the step of applying an electric field to the red blood cell. More preferably, the electric field is from about 0.1 kV/cm to about 10 kV/cm under in vitro conditions. Most preferably, the electric field is applied for between 1 μs and 100 ms.

Where the red blood cell is subject to electrosensitisation, the electrosensitisation of the red blood cell may be performed after the loading of the agent. Alternatively, the electrosensitisation of the red blood cell is performed before the loading of the agent.

The ultrasound may be selected from the group consisting of diagnostic ultrasound, therapeutic ultrasound and a combination of diagnostic and therapeutic ultrasound. Preferably, the applied ultrasound energy source is at a power level of from about 0.05 W/cm² to about 100 W/cm².

As a fourth aspect of the present invention, there is provided a red blood cell delivery vector which has been pre-sensitised such that it is capable of being loaded with a larger amount of agent than a red blood cell which has not been pre-sensitised. Preferably, the red blood cell delivery vector has been pre-sensitised by exposure to an electric field and/or ultrasound. More preferably, the red blood cell delivery vector is sensitised to render it more susceptible to disruption by exposure to a stimulus. Most preferably, the red blood cell delivery vector is loaded with an agent to be delivered.

The agent is preferably selected from a group consisting of a protein, a polypeptide, a peptide, a nucleic acid, a peptide nucleic acid (PNA), a virus, a virus-like particle, a nucleotide, a ribonucleotide, a deoxyribonucleotide, a modified deoxyribonucleotide, a heteroduplex, a nanoparticle, a synthetic analogue of a nucleotide, a synthetic analogue of a ribonucleotide, a modified nucleotide, a modified ribonucleotide, an amino acid, an amino acid analogue, a modified amino acid, a modified amino acid analogue, a steroid, a proteoglycan, a lipid, a fatty acid, an oligosaccharide, a glycoprotein, a carbohydrate, and mixtures, fusions, combinations or conjugates of the above. The agent may be conjugated to, fused to, mixed with or combined with an imaging agent.

We provide, according to a fifth aspect of the present invention, a red blood cell delivery vector obtainable by a method comprising: (a) pre-sensitising a red blood cell by electrosensitising the cell; (b) loading the cell with an agent; and (c) electrosensitising the cell, in which steps (b) and (c) can be performed in any order.

The present invention, in a sixth aspect, provides the use of an electric field and/or ultrasound to increase the efficiency of loading of an agent into a red blood cell.

Preferably, the efficiency of loading of a pre-sensitised cell is 50% or greater, more preferably, 60% or greater, even more preferably, 70% or greater, yet more preferably, 80% or greater. In highly preferred embodiments of the invention, the loading efficiency of a cell pre-sensitised according to our invention is 90% or greater, preferably 95% or 100%. Loading efficiency as used here refers to the percentage of cells which have taken up agent compared with the starting population. Various means may be used for assessing loading efficiency, for example, FACS analysis as described here.

In a seventh aspect of the present invention, there is provided a method of pre-sensitising a red blood cell with an electric field and/or ultrasound such that the amount of agent that is capable of being loaded into the pre-sensitised red blood cell is higher than that which is capable of being loaded into a red blood cell which is not pre-sensitised.

According to an eighth aspect of the present invention, we provide a kit comprising a red blood cell made by a method according to the first, second or third aspect of the invention or a red blood cell delivery vector according to the fourth or fifth aspect of the invention, packaging materials therefor and instructions for use.

We provide, according to a ninth aspect of the invention, a kit comprising a red blood cell, an agent, packaging materials therefor and instructions for use in a method comprising the steps of: (a) pre-sensitising a red blood cell; (b) loading the cell with an agent; (c) electrosensitising the cell; and (d) causing the agent to be released from the sensitised cell by applying ultrasound at a frequency and energy sufficient to cause disruption of the sensitised cell but insufficient to cause disruption of un-sensitised red blood cells, in which steps (b) and (c) can be performed in any order.

There is provided, according to a tenth aspect of the invention, a kit comprising a pre-sensitised red blood cell which is loaded with an agent, packaging materials therefor and instructions for use in a method comprising the steps of: (a) electrosensitising the cell; and (b) causing the agent to be released from the sensitised cell by applying ultrasound at a frequency and energy to cause disruption of the sensitised cell but insufficient to cause disruption of unsensitised red blood cells.

As an eleventh aspect of the invention, we provide a kit comprising a red blood cell delivery vector according to the fifth aspect of the invention, packaging materials therefor and instructions for use comprising the step of causing the agent to be released from the red blood cell delivery vector by applying ultrasound at a frequency and energy to cause disruption of the red blood cell delivery vector but insufficient to cause disruption of un-sensitised red blood cells.

Preferably, the kit further comprises polyethylene glycol. More preferably, the kit further comprises a liquid selected from the group consisting of a buffer, diluent or other excipient. The liquid may be selected from the group consisting of a saline buffer, a physiological buffer, serum and plasma.

We provide, according to a twelfth aspect of the invention, a pharmaceutical composition comprising a red blood cell made by a method according to the first, second or third aspect of the invention or a red blood cell delivery vector according to the fourth or fifth aspect of the invention, together with a pharmaceutically acceptable carrier or diluent.

There is further provided a method and/or a red blood cell delivery vector and/or a kit and/or a pharmaceutical composition substantially as described herein and with reference to the examples and figures.

Furthermore, we provide a device for producing a red blood cell delivery vector of the present invention which device comprises: (a) one or more flow cells and electrosensitisation means; (b) one or more dialysis systems; in which the flow cell is linked to the dialysis system by connecting means capable of allowing the transfer of red blood cells from the flow cell to the dialysis system and vice versa.

Optionally, the device may comprise more than one flow cell, such as two flow cells. The device may also be connected to a collection device such as a blood bag. In a preferred embodiment, the device is also connected to a chromatography and/or filtration stage so that after the final sensitisation procedure, red blood cells are purified and/or the composition of buffer in which the cells are suspended altered.

### BRIEF DESCRIPTION OF THE DRAWINGS

FIG. 1A shows flow cytometry profiles obtained following analysis of human erythrocytes loaded with a fluorescently-labelled oligonucleotide. The X axis represents the fluorescence intensity exhibited by cells in each preparation and the Y-axis represents the number of counts detected by the flow cytometer at a given fluorescence intensity. The control (indicated by RBC) represents the profile exhibited by human erythrocytes which had been placed in contact with the oligonucleotide. Movement of the electroporated peak to the right is indicative of loading of oligonucleotide into the preparation.

FIG. 1B shows flow cytometry profiles obtained following analysis of cell preparations loaded with oligonucleotide using conventional dialysis (-•-) and electropulsing combined with dialysis (Gendel dialysis, -). Control samples consist of human erythrocytes alone (RBC) and erythrocytes together with oligonucleotide without any further treatment (oligo no dialysis). The X-axis represents the fluorescence intensity and the Y-axis represents the number of counts detected at any given fluorescence intensity.

FIG. 2A shows flow cytometry profiles obtained following analysis of human erythroyctes loaded with FITC-labelled antibody by electroporation (-) (exponential decay pulsing). The control sample (RBC+ab) represents the profile exhibited by cells exposed to antibody in the absence of an electroporating pulse. The X-axis represents the fluorescence intensity and the Y-axis represents the number of counts detected at any given fluorescence intensity.

FIG. 2B shows flow cytometry profiles obtained following analysis of human erythrocytes loaded with FITC-labelled antibody by electroporation (-) (square wave pulse). The control sample (RBC+ab) represents the profile exhibited by cells exposed to antibody in the absence of an electroporating pulse. The X-axis represents the fluorescence intensity and the Y-axis represents the number of counts detected at any given fluorescence intensity.

FIG. 3 shows flow cytometry profiles obtained following analysis of human erythroyctes loaded with FITC-labelled antibody by conventional published dialysis (-) and by the dialysis process as described in Example 1 (-). The X-axis represents the fluorescence intensity and the Y-axis represents the number of counts detected at any given fluorescence intensity.

FIG. 4 shows flow cytometry profiles obtained following analysis of human erythroyctes loaded with FITC-labelled antibody using buffer A (A) and buffer B (B). The peak on the left represents the control population prior to loading and that on the right represents the profile obtained following loading. The X-axis represents the fluorescence intensity and the Y-axis represents the number of counts detected at any given fluorescence intensity.

FIG. 5 shows ultrasound-mediated release of anti von Willebrand factor antibody from sensitised human erythrocytes in perfused rat kidney.

FIG. 6 shows the stability of cellular integrity (cell numbers) and ultrasound sensitivity during storage at 4° C. Cells were loaded with FITC labelled antibody using a process comprising pre-sensitisation/hypotonic dialysis/electrosensitisation (ES-HD-ES) and at the indicated times cell numbers ( ) were determined by direct counting. The percentage of cells that lysed following exposure to ultrasound was also determined ( ) for each sample. The X-axis represents the time in days, the left Y-axis represents the percentage of cells remaining intact and the right Y-axis represents the percentage lysis observed following exposure to ultrasound.

FIG. 7 shows flow cytometry profiles obtained following analysis of payload retention in samples stored at time zero and 30 days at 4° C. The peak on the right in the 30 day old sample exhibited a similar fluorescence intensity to that analysed at time zero indicating that the payload was retained throughout the storage period. The X-axis represents the fluorescence intensity and the Y-axis represents the number of counts detected at any given fluorescence intensity.

FIG. 8 is a graph showing the ultrasound-mediated release of antibody from the erythrocyte vehicle. X-axis: power density (W/cm²), left hand Y-axis: μg of anti-vWF released (per 7×10⁷ cells treated by ultrasound); right hand Y-axis: percentage of cells lysed by ultrasound. Filled squares represent μg antibody, loaded cells; filled triangles represent μg antibody, control cells; open squares represent % lysis, loaded cells; open triangles represent % lysis, control cells.

FIG. 9 is a graph showing ultrasound mediated release of β-galactosidase from the erythrocyte vehicle. X-axis: power density (W/cm²), left hand Y-axis: percentage lysis; right hand Y-axis: % relative enzyme release. Filled squares represent control lysis, filled triangles represent sample lysis and filled diamonds represent sample release (i.e., release of enzyme).

FIG. 10 is a graph showing ultrasound-mediated release of oligonucleotide from the erythrocyte vehicle. X-axis: power density (W/cm²), left hand Y-axis: percentage cell lysis; right hand Y-axis: % oligo release. Filled squares represent control cell lysis, filled triangles represent cell lysis and filled diamonds represent % oligo released.

FIG. 11 shows flow cytometry profiles obtained following analysis of human erythrocytes loaded with fluorescein-labelled anti-rat IgG using (i) electrosensitisation (pre-sensitisation)-hypoosmotic dialysis-electrosensitisation protocol (ES+HD+ES; - - - ; “pre-sensitisation”), (ii) hypoosmotic dialysis alone (HD; -;”dialysis”) or (iii) a sonoporation-hypoosmotic dialysis-electrosensitisation (SP+HD+ES; . . . ; “sonoporation”) protocol. The X-axis represents the fluorescence intensity and the Y-axis represents the number of counts detected at any given fluorescence intensity.

FIG. 12 shows ultrasound-mediated release of antibody payload (anti-von Willebrand factor antibody) from loaded and sensitised human cells diluted in normal human cells at 40% hematocrit. Continuous wave ultrasound at 5 W/cm² is used. X-axis: ultrasound exposure time (minutes); Y-axis: antibody payload released (%). The target cells were circulated through a system in which the temperature was maintained at 37° C. and the flow rate during exposure was 14.5 ml/min. Gray bars: control; black bars: test.

### DETAILED DESCRIPTION OF THE INVENTION

Sensitisation/pre-sensitisation

In the method of the present invention, cells are subject to at least two sensitisation steps, one of which must be performed prior to, or concomitant with, the loading step, preferably prior to the loading step. For this reason, the first sensitisation step is referred to herein as a pre-sensitisation step. The purpose of the pre-sensitisation step is to enhance the loading of the agent, although an increase in sensitivity to lysis may also be achieved. The additional sensitisation steps may be performed at any stage in the process after the pre-sensitisation step. The purpose of the additional sensitisation step or steps is to increase the sensitivity of the cells to ultrasound.

In two particular embodiments that are exemplified herein, a second sensitisation step is carried out either after the pre-sensitisation step but prior to dialysis loading, or after dialysis loading. Further sensitisation steps may be performed as required.

Generally, the sensitisation steps and the loading step are temporally separated. For example, cells are typically allowed to rest in buffer, such as PBS/Mg/glucose buffer, for at least 30 mins, preferably at least 60 mins, after a pre-sensitisation step to allow the cells to recover prior to loading or further sensitisation steps. It may be desirable to allow cells to rest for several hours, such as overnight, after the loading step.

The pre-sensitisation step increases the efficiency of loading of an agent into a red blood cell, compared to a red blood cell which has not been subject to pre-sensitisation. The pre-sensitisation may take the form of an electrosensitisation step, as described below. Alternatively, or in addition, the pre-sensitisation may be effected by the use of ultrasound, as described below and shown in the Examples. Other methods may be used to pre-sensitise cells and enhance loading efficiency. For example, electromagnetic radiation such as microwaves, radio waves, gamma rays and X-rays may be used. In addition, the use of chemical agents such as DMSO and pyrrolidinone may be envisaged. Furthermore, thermal energy may be imparted on the red blood cells to pre-sensitise them. This may be achieved by raising the temperature of the red blood cells by conventional means, by heat shock, or by the use of microwave irradiation. In general, any method which allows pores to be formed on the surface membrane of a red blood cell is a suitable candidate for use as a pre-sensitisation step.

Preferably, the sensitisation step comprises an electrosensitisation procedure as described next. We have found that the efficiency of sensitisation for given electrical parameters varies depending on the cell density and it may therefore be necessary to perform a titration of cell density and or electrical parameters to establish the optimum concentration. By way of guidance, we have found that cells sensitised at a density of about 6-8×10⁸ cells/ml had good sensitivity to ultrasound.

Electrosensitisation

The present invention encompasses the use of an electric field for sensitising a red blood cell to a disruptive stimulus, such as ultrasound. Electrosensitisation describes preferably using an electrical field to sensitize the cell, but other forms of electromagnetic energy may also be used as a means of rendering the cells more susceptible to lysis. Electrosensitization also may comprise pre-sensitising red blood cells using an electric field.

The terms “sensitized” and “electrosensitisation” encompasses the destabilisation of cells without causing fatal damage to the cells. For “elecrosensitization”, a momentary exposure of a cell to one or more pulses at high electric field strength results in membrane destabilisation. The strength of the electric field is adjusted up or down depending upon the resilience or fragility, respectively, of the cells being loaded and the ionic strength of the medium in which the cells are suspended. The sensitized cells display a hypersensitivity relative to unsensitized cells, to for example, ultrasound or light energy.

Electrosensitisation typically involves the use of electric fields which do not possess sufficient energy to electroporate the cells. Electroporation, which facilitates the passage of agents into the cell without significant loss of cellular contents or cell viability, is well known in the art, and apart from the energy levels involved is similar to electrosensitisation. Indeed, cells which are electroporated become electrosensitised. However, electrosensitisation may be carried out at energy levels which are insufficient to electroporate the cell and permit the passage of substances through the cell wall and/or cell membrane. In a highly preferred embodiment of the present invention, electrosensitisation of the red blood cells is carried out at these energy levels.

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both supplied by the BTX Division of Genetronics, Inc (see U.S. Pat. No 5,869,326).

These known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1 kV/cm, of about 100 μs duration. Such a pulse may be generated, for example, in known applications of the Electro Square Porator T820.

Electrosensitisation may be performed in a manner substantially identical to the procedure followed for electroporation, with the exception that lower electric field strengths may be used, as set forth below.

In a preferred aspect of the present invention, the electric field has a strength of from about 0.1 kV/cm to about 10 kV/cm under in vitro conditions, more preferably from about 1.5 kV/cm to about 4.0 kV/cm under in vitro conditions. Most preferably, the electric field strength is about 3.625 kV/cm under in vitro conditions.

Preferably the electric field has a strength of from about 0.1 kV/cm to about 10 kV/cm under in vivo conditions (see WO97/49450). More preferably, the electric field strength is about 3.625 kV/cm under in vitro conditions.

Preferably the application of the electric field is in the form of multiple pulses such as double pulses of the same strength and capacitance or sequential pulses of varying strength and/or capacitance. A preferred type of sequential pulsing comprises delivering a pulse of less than 1.5 kV/cm and a capacitance of greater than 5 F, followed by a pulse of greater than 2.5 kV/cm and a capacitance of less than 2 F, followed by another pulse of less than 1.5 kV/cm and a capacitance of greater than 5 F. A particular example is 0.75 kV/cm, 10 F; 3.625 kV/cm, 1 F and 0.75 kV/cm, 10 F.

Preferably the electric pulse is delivered as a waveform selected from an exponential wave form, a square wave form and a modulated wave form.

As used herein, the term “electric pulse” includes one or more pulses at variable capacitance and voltage and including exponential and/or square wave and/or modulated wave forms.

Other electroporation procedures and methods employing electroporation devices are widely used in cell culture, and appropriate instrumentation is well known in the art.

In a particularly preferred embodiment, the following electrosensitisation protocol is used. Cells are suspended in PBS to yield concentrations of about 6-8×10⁸ cells/ml and 0.8 ml aliquots are dispensed into sterile electroporation cuvettes (0.4 cm electrode gap) and retained on ice for 10 min. Cells are then exposed to an sensitisation strategy involving delivery of two electric pulses (field strength=3.625 kV/cm at a capacitance of 1 μF) using a BioRad Gene Pulser apparatus. Cells are immediately washed with PBS containing MgCl₂ (4 mM) (PBS/Mg) and retained at room temperature for at least 30 min in the PBS/Mg buffer at a concentration of 7×10⁸ cells/ml to facilitate re-sealing. Optionally, cells are subsequently washed and suspended at a concentration of 7×10⁸ cells/ml in PBS/Mg containing 10 mM glucose (PBS/Mg/glucose) for at least 1 hour.

Pre-sensitisation Using Ultrasound

As noted above, ultrasound may be used to pre-sensitise red blood cells. Such use of ultrasound is also referred to herein as “sonoporation”. Exposure of red blood cells to ultrasound is believed to result in non-destructive and transient membrane poration (Miller et al, 1998, Ultrasonics 36, 947-952).

As used herein, the term “ultrasound” refers to a form of energy which consists of mechanical vibrations the frequencies of which are so high they are above the range of human hearing. The lower frequency limit of the ultrasonic spectrum may generally be taken as about 20 kHz. Most diagnostic applications of ultrasound employ frequencies in the range 1 and 15 MHz (from Ultrasonics in Clinical Diagnosis. Edited by PNT Wells, 2nd. Edition, Publ. Churchill Livingstone [Edinburgh, London & NY, 1977].

Ultrasound has been used in both diagnostic and therapeutic applications. When used as a diagnostic tool (“diagnostic ultrasound”), ultrasound is typically used in an energy density range of up to about 100 mW/cm (FDA recommendation), although energy densities of up to 750 mW/cm² have been used. In physiotherapy, ultrasound is typically used as an energy source in a range up to about 3 to 4 W/cm² (WHO recommendation). In other therapeutic applications, higher intensities of ultrasound may be employed, for example, HIFU at 100 W/cm² up to 1 kW/cm² (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al., 1998 Journal of Magnetic Resonance Imaging Vol.8, No.1, pp.136-142. Another form of focused ultrasound is high intensity focused ultrasound (HIFU) which is reviewed by Moussatov et al. in Ultrasonics, 1998 Vol.36, No.8, pp.893-900 and TranHuuHue et al. in Acustica, 1997, Vol.83, No.6, pp.1103-1106.

Preferably, the red blood cells are pre-sensitised by exposure to ultrasound that has an energy density in the therapeutic range. In a highly preferred embodiment, treatment is at 2.5 W/cm² for 5 min using a 1 MHz ultrasound head. This combination is however not intended to be limiting. Indeed, various combinations of frequency, energy density and exposure time may be used to pre-sensitise the red blood cells so that their loading efficiency is increased.

Loading

As used herein, the term “loading” refers to introducing into a red blood at least one agent. The agent may be loaded by becoming internalised by, affixed to the surface of, or anchored into the plasma membrane of a red blood cell. Where the agent is affixed or anchored to the plasma membrane, loading may be achieved by cross-linking the agent to any cell surface molecule. Alternatively, the agent may be conjugated to or fused with an antibody specific for a cell surface molecule.

Loading of a red blood cell with more than one agent may be performed such that the agents are loaded individually (in sequence) or together (simultaneously or concurrently). Loading is generally performed in a separate procedure to the “sensitising” procedure. The agents may be first admixed at the time of contact with the red blood cells or prior to that time.

According to the present invention, red blood cells are loaded either after the pre-sensitisation procedure or after one or more sensitisation procedures, preferably after the cells have rested. In this embodiment, the loading may be performed by any desired technique. Accordingly, the present invention encompasses the sensitisation of a pre-sensitised and loaded cell. It also encompasses the loading of a pre-sensitised and subsequently sensitised cell.

The loading may be performed by a procedure selected from the group consisting of electroporation, iontophoresis, sonoporation, microinjection, calcium precipitation, membrane intercalation, microparticle bombardment, lipid-mediated transfection, viral infection, osmosis, osmotic pulsing, osmotic shock, diffusion, endocytosis, phagocytosis, crosslinking to a red blood cell surface component, chemical crosslinking, mechanical perforation/restoration of the plasma membrane by shearing, single-cell injection or a combination thereof.

Sonoporation as a method for loading an agent into a cell is disclosed in, for example, Miller et al (1998), Ultrasonics 36, 947-952.

Iontophoresis uses electrical current to activate and to modulate the diffusion of a charged molecule across a biological membrane, such as the skin, in a manner similar to passive diffusion under a concentration gradient, but at a facilitated rate. In general, iontophoresis technology uses an electrical potential or current across a semipermeable barrier. By way of example, delivery of heparin molecules to patients has been shown using iontophoresis, a technique which uses low current (d.c.) to drive charged species into the arterial wall. The iontophoresis technology and references relating thereto is disclosed in WO 97/49450.

In a highly preferred embodiment, the red blood cell is pre-sensitised by electrosensitisation, and loaded using osmotic shock. If more than one agent is employed, the same or a different technique may be used to load the second agent into the red blood cell.

Preferably the red blood cells of the present invention are pre-sensitised, sensitised and loaded in vitro or ex-vivo.

Preferably loading is carried out by an osmotic shock procedure. The term “osmotic shock” is intended herein to be synonymous with the term “hypotonic dialysis” or “hypoosmotic dialysis”.

A preferred osmotic shock/hypotonic dialysis method is described in the Examples and is based on the method described in Eichler et al., 1986, Res. Exp. Med. 186: 407-412. This preferred method is as follows. Washed red blood cells are suspended in 1 ml of PBS (150 mM NaCl, 5 mM K₂HPO₄/KH₂PO₄; pH 7.4) to obtain a hematocrit of approximately 60%. The suspension is placed in dialysis tubing (molecular weight cut-off 12-14,000; Spectra-Por; prepared as outlined below) and swelling of cells obtained by dialysis against 100 ml of 5 mM K₂HPO₄/KH₂PO₄, pH 7.4 for 90 minutes at 4° C. Resealing is achieved by subsequent dialysis for 15 minutes at 37° C. against 100 ml of PBS containing 10 mM glucose. Cells are then washed in ice cold PBS containing 10 mM glucose using centrifugation.

Other osmotic shock procedures include the method described in U.S. Pat. No. 4,478,824. That method involves incubating a packed red blood cell fraction in a solution containing a compound (such as dimethyl sulphoxide (DMSO) or glycerol) which readily diffuses into and out of cells, rapidly creating a transmembrane osmotic gradient by diluting the suspension of red blood cell in the solution with a near-isotonic aqueous medium. This medium contains an anionic agent to be introduced (such as inosine monophosphate or a phosphorylated inositol, for example inositol hexaphosphate) which may be an allosteric effector of haemoglobin, thereby causing diffusion of water into the cells with consequent swelling thereof and increase in permeability of the outer membranes of the cells. This increase in permeability is maintained for a period of time sufficient only to permit transport of the anionic agent into the cells and diffusion of the readily-diffusing compound out of the cells. This method is of limited effectiveness where the desired agent to be loaded into cells is not anionic, or is anionic or polyanionic but is not present in the near-isotonic aqueous medium in sufficient concentration to cause the needed increase in cell permeability without cell destruction.

U.S. Pat. No. 4,931,276 and WO 91/16080 also disclose methods of loading red blood cells with selected agents using an osmotic shock technique. Therefore, these techniques can be used to enable loading of red blood cells in the present invention.

Effective agents which may advantageously be loaded into red blood cells using the modified method provided in U.S. Pat. No. 4,931,276 include peptides, purine analogues, pyrimidine analogues, chemotherapeutic agents and antibiotic agents. These agents frequently present drug delivery problems. Specific compounds include but are not limited to tryptophan, phenylalanine and other water-soluble amino acid compounds. Several derivatives of the unnatural analogues of the nucleic acid bases adenine, guanine, cytosine and thymine are well known as useful therapeutic agents, e.g. 6-mercaptopurine (6MP) and azathioprine, which are commonly used as immunosuppressants and inhibitors of malignant cell growth, and azidothymidine (AZT) and analogues thereof which are useful as anti-viral agents, particularly in the treatment of AIDS. It has been shown that the action of these unnatural base derivatives is dependent on intra-cellular conversion thereof to phosphorylated forms (Chan et al., 1987, Pharmacotherapy, 7: 165;14 177; also Mitsuya et al., 1986, Proc. Natl. Acad. Sci. U.S.A., 83: 1911-1915).

An alternative osmotic shock procedure is described in U.S. Pat. No. 4,931,276 which is incorporated herein by reference.

Alternatively, loading may be carried out by a microparticle bombardment procedure. Microparticle bombardment entails coating gold particles with the agent to be loaded, dusting the particles onto a 22 calibre bullet, and firing the bullet into a restraining shield made of a bullet-proof material and having a hole smaller than the diameter of the bullet, such that the gold particles continue in motion toward cells in vitro and, upon contacting these cells, perforate them and deliver the payload to the cell cytoplasm.

It will be appreciated by one skilled in the art that combinations of methods may be used to facilitate the loading of a red blood cell with agents of interest according to the invention. Likewise, it will be appreciated that a first and second agent, may be loaded concurrently or sequentially, in either order, into a red blood cell in any method of the present invention.

As would be apparent to one of skill in the art, any one or more of the above techniques can be used to load red blood cells for use in the invention, either prior to, simultaneously with, separate from or in sequence to the sensitisation procedure. For example, U.S. Pat. No. 4,224,313 discloses a process for preparing a mass of loaded cells suspended in a solution by increasing the permeability of the cell membranes by osmotic pressure or an electric field, or both, loading agents by passage from a solution through the membranes of increased permeability, restoring the original permeability by sealing the membranes by regeneration effect, and separating the cells from the solution in which they were suspended. In that procedure, the agents in solution which are to be loaded include i) a pharmaceutical substance which reacts chemically or physically with substances in the extracellular milieu and which, when loaded into the cell, would prematurely destroy the cell membranes, and ii) at least one blood-compatible sugar and protein capable of providing hydrogen bridge bonding- or of entering into covalent bonds with the pharmaceutical substance, thereby inhibiting the reaction of the pharmaceutical substance with the cell membranes.

It will be appreciated by one skilled in the art that combinations of methods may be used to facilitate the loading of a red blood cell with agents of interest according to the invention. Likewise, it will be appreciated that a first and second agent, may be loaded concurrently or sequentially, in either order, into a red blood cell in any method of the present invention.

The concentration of agent used in the loading procedure may need to be optimised.

For example, we have shown that an FITC-IgG antibody achieves good loading at concentrations of 0.1 mg/ml to 2 mg/ml.

Preferably loading takes place over a period of at least 30 mins, more preferably about 90 mins.

Selective Release Using Ultrasound

According to the invention, agents which are loaded into a red blood cell are released from the red blood cell and into their surroundings, in this case at or into the target site, tissue or cell, by the application of ultrasound directed at a target site, tissue and/or cell. Furthermore, the agent may be delivered to the target site by application of ultrasound to vessels, for example, blood vessels, feeding the target site. A general discussion on ultrasound, including different types of ultrasound (for example, diagnostic, therapeutic and focussed ultrasound), is presented above.

Preferably, a combination of diagnostic ultrasound and a therapeutic ultrasound is employed to effect selective release. This combination is not intended to be limiting, however, and the skilled reader will appreciate that any variety of combinations of ultrasound may be used. Additionally, the energy density, frequency of ultrasound, and period of exposure may be varied. What is important is that the application of ultrasound is able to selectively disrupt the sensitised red blood cells to effect release of agent, without substantially disrupting or damaging endogenous red blood cells.

Preferably the ultrasound is applied to a target cell or target tissue with sufficient strength to disrupt loaded and sensitised red blood cells but without damaging the target tissue or surrounding tissues. In this context, the term “damage or damaging” does not include a transient permeabilisation of the target site by the ultrasound energy source. Such a permeabilisation may facilitate uptake of the released payload at the target site.

Preferably the exposure to an ultrasound energy source is at a power density of from about 0.05 to about 100 Wcm⁻². Even more preferably, the exposure to an ultrasound energy source is at a power density of from about 1 to about 15 Wcm⁻².

Preferably the exposure to an ultrasound energy source is at a frequency of from about 0.015 to about 10.0 MHz. More preferably the exposure to an ultrasound energy source is at a frequency of from about 0.02 to about 5.0 MHz.

Preferably the exposure is for periods of from about 10 milliseconds to about 60 minutes. More preferably the exposure is for periods of from about 1 second to about 5 minutes. Depending on the amount of agent which it is desired to release, however, the exposure may be for a longer duration, for example, for 15 minutes.

Particularly preferably the patient is exposed to an ultrasound energy source at an acoustic power density of from about 0.05 Wcm⁻² to about 10 Wcm⁻² with a frequency ranging from about 0.015 to about 10 MHz (see WO 98/52609). However, alternatives are also possible, for example, exposure to an ultrasound energy source at an acoustic power density of above 100 Wcm⁻², but for reduced periods of time, for example, 1000 Wcm⁻² for periods in the millisecond range or less.

Use of ultrasound is advantageous as, like light, it can be focused accurately on a target. Moreover, ultrasound is advantageous as it can be focussed more deeply into tissues unlike light. It is therefore better suited to whole-tissue penetration (such as but not limited to a lobe of the liver) or whole organ (such as but not limited to the entire liver or an entire muscle, such as the heart) delivery of agents according to the present invention. In addition, ultrasound may induce a transient permeabilisation of the target site so that uptake of a released payload is facilitated at the target site. Another important advantage is that ultrasound is a non-invasive stimulus which is used in a wide variety of diagnostic and therapeutic applications. By way of example, ultrasound is well known in medical imaging techniques and, additionally, in orthopaedic therapy. Furthermore, instruments suitable for the application of ultrasound to a subject vertebrate are widely available and their use is well known in the art.

In methods of the invention, release of the agent is effected by exposure of red blood cells either in vitro or ex-vivo to an effective amount of a diagnostic ultrasound energy source or a therapeutic ultrasound energy source as described in U.S. Pat. No. 5,558,092 and WO94/28873. The agent, which is released from a red blood cell for use in the present invention may be referred to as the “payload” of that cell.

Preferably the agent is released from the red blood cell by treatment of a target site, tissue or cell with ultrasound.

“Release” is not intended to require complete release of the agent from the red blood cell or red blood cell vector, i.e, that all of the agent physically separates from the red blood cell or cellular particles, or that all of the red blood cells that carry the agent are disrupted. Substantial or selective release, as described by this invention, would be a release of the agent at an appropriate location at a concentration and amount acceptable and consistent with achieving the goals of this invention. The selective release of the agent at the target site can be determined by observing among other things, a) the amount which has been released at the target site, tissue or cell and b) its effect on the target site, tissue or cell, the latter determining whether its delivery should increase, decrease or be discontinued.

Blood Cells

In one embodiment of the present invention, the red blood cells which may be loaded and administered to a vertebrate according to the invention are ideally obtained from the intended recipient individual prior to the procedure so as to ensure complete immuno-compatibility. Alternatively, cells are obtained from a second individual of the same species as the recipient; in such a case, the second individual must share the blood type of the intended recipient or must have an immuno-neutral blood type, such as type O in humans. Alternatively, the red blood cell may have its immunological determinants masked by a substance such as PEG and/or modified, for example by one or more enzymes.

As used herein, the term “red blood cell” refers to a living, enucleate red blood cell (i.e., a mature erythrocyte) of a vertebrate.

Preferably the red blood cell is a mammalian red blood cell, advantageously a human red blood cell. As used herein, the term “mammal” refers to a member of the class Mammalia including, but not limited to, a rodent, lagomorph, pig or primate. Preferably, the mammal is a human.

As used herein the term “introducing” includes but is not limited to the administration of a red blood cell and/or an agent into a vertebrate.

As used herein in reference to administration of an agent to a vertebrate, the term “introducing” includes but is not limited to causing the agent to enter the circulatory system of the vertebrate by transfusion or to infusing an agent to a target site. It is contemplated that a hollow needle, such as a hypodermic needle or cannula, is inserted through the wall of a blood vessel (e.g., a vein or artery) and the red blood cell is either injected using applied pressure or allowed to diffuse or otherwise migrate into the blood vessel. It is understood that the diameter of the needle is sufficiently large and the pressure sufficiently light to avoid damage of the cell by shear forces. Preferably, introduction of a red blood cell into a vertebrate in a method of the invention is intra-arterial or intravenous. Methods of blood cell transfusion are well known in the art.

As used herein, the term “red blood cell delivery vector” means a red blood cell that has been electrosensitised and loaded with one or more agents according to the methods of the invention and can be used to deliver the agent to a vertebrate. The red blood cell delivery vector is typically made to release the agent at a site of interest in the vertebrate using ultrasound as described above.

Immunocompatibility

“Immunocompatibility” as used in this invention is intended to be interpreted broadly. It contemplates both the activation or inhibition of an immune response, whether that response is humoral, or cell mediated, or otherwise. It is further intended to refer to generalized interactions with immune cells or immune receptors irrespective of whether an immune response is generated from said interaction.

It will be apparent to one skilled in the art that it may be desirable to affect the immunocompatibility of the sensitized red blood cells described by this invention. This would be particularly contemplated when said cells are intended to be used in the treatment of disease or delivery or an agent to a site in the animal, or to immune cells themselves. It is contemplated that this may involve rendering the red blood cell more immunogenic so that, for example, the effects of the agent can be enhanced by, or affect or initiate an immune response in an animal. It is also contemplated that it may be desirable to render said red blood cell less immunogenic so that, by example, said sensitized red blood cells or agents they further comprise, do not provoke, or are not sequestered by the immune system. Methods of affecting immunoregulation in an animal are well described in the art, and their selection and use will be apparent to one skilled in the art. By way of non-limiting example, the following approaches are specifically contemplated for reducing the immunogenicity of the sensitized red blood cell as contemplated by this invention.

The loaded red blood cell vehicles may be coated with an agent which masks cell surface antigens. For example, PEGylated red blood cells evade the host immune response and thereby enjoy prolonged circulation. According to one such method, methoxy (polyethylene glycol), or mPEG, is covalently bound to red blood cells (Scott et al., 1997, Proc. Natl. Acad. Sci. U.S.A., 94: 7566-7571). This procedure has been shown to result in a loss of ABO blood group reactivity and inhibition of phagocytic destruction by monocytes; in addition, the survival of mPEG treated sheep red blood cells transfused into mice is increased 360-fold over that of untreated control cells (Scott et al., 1997, supra).

A second coating which may be useful in the invention is one which comprises distearoyl-phosphatidylethanolamine (DSPE)-conjugated PEG (Du et al., 1997, Biochim. Biophys. Acta-Biomembranes, 1326: 236-248). When applied as a monolayer film to a glass plate, DSPE-PEG inhibits protein adsorption and cell adhesion to the glass plate (Du et al., 1997, supra).

Agent

As used herein, the term “agent” includes but is not limited to an atom or molecule, wherein a molecule may be inorganic or organic, a biological effector molecule and/or a nucleic acid encoding an agent such as a biological effector molecule, a protein, a polypeptide, a peptide, a nucleic acid, a peptide nucleic acid (PNA), a virus, a virus-like particle, a nucleotide, a ribonucleotide, a synthetic analogue of a nucleotide, a synthetic analogue of a ribonucleotide, a modified nucleotide, a modified ribonucleotide, an amino acid, an amino acid analogue, a modified amino acid, a modified amino acid analogue, a steroid, a proteoglycan, a lipid, a fatty acid and a carbohydrate. An agent may be in solution or in suspension (e.g., in crystalline, colloidal or other particulate form). The agent may be in the form of a monomer, dimer, oligomer, etc, or otherwise in a complex. The term “agent” further contemplates regions of an agent which display alone or in combination, biological activity, e.g., the active site of an enzyme, or active domains of a protein, or functional groups of a molecule, or any combination of the above.

The agent may comprise an imaging agent or region therof, by which term is meant an agent which may be detected, whether in vitro in the context of a tissue, organ or organism in which the agent is located. The imaging agent may emit a detectable signal, such as light or other electromagnetic radiation. The imaging agent may be a radio-isotope as known in the art, for example ³²P or ³⁵S or ⁹⁹Tc, or a molecule such as a nucleic acid, polypeptide, or other molecule as explained below conjugated with such a radio-isotope. The imaging agent may be opaque to radiation, such as X-ray radiation. The imaging agent may also comprise a targeting means by which it is directed to a particular cell, tissue, organ or other compartment within the body of an animal. For example, the agent may comprise a radiolabelled antibody specific for defined molecules, tissues or cells in an organism.

The imaging agent may be combined with, conjugated to, mixed with or combined with, any of the agents disclosed herein. Combinations of agents with multiple of overlapping specificities or utilities are clearly contemplated by this invention.

It will be appreciated that it is not necessary for a single agent to be used, and that it is possible to load two or more agents for into the vehicle. Accordingly, the term “agent” also includes mixtures, fusions, combinations and conjugates, of atoms, molecules etc as disclosed herein. For example, an agent may include but is not limited to: a nucleic acid combined with a polypeptide; two or more polypeptides conjugated to each other; a protein conjugated to a biologically active molecule (which may be a small molecule such as a prodrug); or a combination of a biologically active molecule with an imaging agent.

As used herein, the term “biological effector molecule” or “biologically active molecule” refers to an agent that has activity in a biological system, including, but not limited to, a protein, polypeptide or peptide including, but not limited to, a structural protein, an enzyme, a cytokine (such as an interferon and/or an interleukin) an antibiotic, a polyclonal or monoclonal antibody, or an effective part thereof, such as an Fv fragment, which antibody or part thereof may be natural, synthetic or humanised, a peptide hormone, a receptor, a signalling molecule or other protein; a nucleic acid, as defined below, including, but not limited to, an oligonucleotide or modified oligonucleotide, an antisense oligonucleotide or modified antisense oligonucleotide, cDNA, genomic DNA, an artificial or natural chromosome (e.g. a yeast artificial chromosome) or a part thereof, RNA, including mRNA, tRNA, rRNA or a ribozyme, or a peptide nucleic acid (PNA); a virus or virus-like particles; a nucleotide or ribonucleotide or synthetic analogue thereof, which may be modified or unmodified; an amino acid or analogue thereof, which may be modified or unmodified; a non-peptide (e.g., steroid) hormone; a proteoglycan; a lipid; or a carbohydrate. If the biological effector molecule is a polypeptide, it may be loaded directly into a red blood cell of the invention; alternatively, a nucleic acid molecule bearing a sequence encoding the polypeptide, which sequence is operatively linked to transcriptional and translational regulatory elements active in a cell at the target site, may be loaded. Small molecules, including inorganic and organic chemicals, are also of use in the present invention. In a particularly preferred embodiment of the invention, the biologically active molecule is a pharmaceutically active agent, for example, an isotope.

Particularly useful classes of biological effector molecules include, but are not limited to, antibiotics, anti-inflammatory drugs, angiogenic or vasoactive agents, growth factors and cytotoxic agents (e.g., tumour suppressers). Cytotoxic agents of use in the invention include, but are not limited to, diptheria toxin, Pseudomonas exotoxin, cholera toxin, pertussis toxin, and the prodrugs peptidyl-p-phenylenediamine-mustard, benzoic acid mustard glutamates, ganciclovir, 6-methoxypurine arabinonucleoside (araM), 5-fluorocytosine, glucose, hypoxanthine, methotrexate-alanine, N-[4-(a-D-galactopyranosyl) benyloxycarbonyl]-daunorubicin, amygdalin, azobenzene mustards, glutamyl p-phenylenediamine mustard, phenolmustard-glucuronide, epirubicin-glucuronide, vinca-cephalosporin, phenylenediamine mustard-cephalosporin, nitrogen-mustard-cephalosporin, phenolmustard phosphate, doxorubicin phosphate, mitomycin phosphate, etoposide phosphate, palytoxin-4-hydroxyphenyl-acetamide, doxorubicin-phenoxyacetamide, melphalan-phenoxyacetamide, cyclophosphamide, ifosfamide or analogues thereof. If a prodrug is loaded in inactive form, a second biological effector molecule may be loaded into the red blood cell of the present invention. Such a second biological effector molecule is usefully an activating polypeptide which converts the inactive prodrug to active drug form, and which activating polypeptide is selected from the group that includes, but is not limited to, viral thymidine kinase (encoded by Genbank Accession No. J02224), carboxypeptidase A (encoded by Genbank Accession No. M27717), α-galactosidase (encoded by Genbank Accession No. M13571), β-glucuronidase (encoded by Genbank Accession No. M15182), alkaline phosphatase (encoded by Genbank Accession No. J03252 J035 12), or cytochrome P-450 (encoded by Genbank Accession No. D00003 N00003), plasmin, carboxypeptidase G2, cytosine deaminase, glucose oxidase, xanthine oxidase, β-glucosidase, azoreductase, t-gutamyl transferase, β-lactamase, or penicillin amidase. Preferably, the polypeptide capable of activating a prodrug is DT diaphorase. Either the polypeptide or the gene encoding it may be loaded; if the latter, both the prodrug and the activating polypeptide may be encoded by genes on the same recombinant nucleic acid construct.

Preferably the biological effector molecule is selected from the group consisting of a protein, a polypeptide, a peptide, a nucleic acid, a virus, a virus-like particle, a nucleotide, a ribonucleotide, a synthetic analogue of a nucleotide, a synthetic analogue of a ribonucleotide, a modified nucleotide, a modified ribonucleotide, an amino acid, an amino acid analogue, a modified amino acid, a modified amino acid analogue, a steroid, a proteoglycan, a lipid and a carbohydrate or a combination thereof (e.g., chromosomal material comprising both protein and DNA components or a pair or set of effectors, wherein one or more convert another to active form, for example catalytically).

The present invention advantageously employs agents which are not able to diffuse through an intact erythrocyte cell wall by passive or active means. However, the delivery of agents which diffuse at a certain rate through the erythrocyte cell wall is contemplated, particularly where increased delivery of the agent at a particular time or location is desirable. Increased delivery may be achieved by ultrasound administation at the appropriate time or location.

The agents, including biological effector molecules, may also be delivered into cells as fusions (for example, protein or polypeptide fusions) or conjugates with a protein capable of crossing the plasma membrane and/or the nuclear membrane. Preferably, the agent/biological effector molecule is fused or conjugated to a domain or sequence from such a protein responsible for the translocational activity. Preferred translocation domains and sequences include domains and sequences from the HIV-1-trans-activating protein (Tat), Drosophila Antennapedia homeodomain protein and the herpes simplex-1 virus VP22 protein. By this means, the agent/biological effector molecule is able to enter the cell or its nucleus when released in the vicinity of the cell using the methods described herein.

Exogenously added HIV-1-trans-activating protein (Tat) can translocate through the plasma membrane and to reach the nucleus to transactivate the viral genome. Translocational activity has been identified in amino acids 37-72 (Fawell et al., 1994, Proc. Natl. Acad. Sci. U.S.A. 91, 664-668), 37-62 (Anderson et al., 1993, Biochem. Biophys. Res. Commun. 194, 876-884) and 49-58 (having the basic sequence RKKRRQRRR) of HIV-Tat. Vives et al. (1997), J Biol Chem 272, 16010-7 identified a sequence consisting of amino acids 48-60 (CGRKKRRQRRRPPQC), which appears to be important for translocation, nuclear localisation and trans-activation of cellular genes. Intraperitoneal injection of a fusion protein consisting of -galactosidase and a HIV-TAT protein transduction domain results in delivery of the biologically active fusion protein to all tissues in mice (Schwarze et al., 1999, Science 285, 1569-72)

The third helix of the Drosophila Antennapedia homeodomain protein has also been shown to possess similar properties (reviewed in Prochiantz, A., 1999, Ann N Y Acad Sci, 886, 172-9). The domain responsible for translocation in Antennapedia has been localised to a 16 amino acid long peptide rich in basic amino acids having the sequence RQIKIWFQNRRMKWKK (Derossi, et al., 1994, J Biol Chem, 269, 10444-50). This peptide has been used to direct biologically active substances to the cytoplasm and nucleus of cells in culture (Theodore, et al., 1995, J. Neurosci 15, 7158-7167). Cell internalization of the third helix of the Antennapedia homeodomain appears to be receptor-independent, and it has been suggested that the translocation process involves direct interactions with membrane phospholipids (Derossi et al., 1996, J Biol Chem, 271, 18188-93). The VP22 tegument protein of herpes simplex virus is capable of intercellular transport, in which VP22 protein expressed in a subpopulation of cells spreads to other cells in the population (Elliot and O'Hare, 1997, Cell 88, 223-33). Fusion proteins consisting of GFP (Elliott and O'Hare, 1999, Gene Ther 6, 149-51), thymidine kinase protein (Dilber et al., 1999, Gene Ther 6, 12-21) or p53 (Phelan et al., 1998, Nat Biotechnol 16, 440-3) with VP22 have been targeted to cells in this manner.

Particular domains or sequences from proteins capable of translocation through the nuclear and/or plasma membranes may be identified by mutagenesis or deletion studies. Alternatively, synthetic or expressed peptides having candidate sequences may be linked to reporters and translocation assayed. For example, synthetic peptides may be conjugated to fluoroscein and translocation monitored by fluorescence microscopy by methods described in Vives et al. (1997), J Biol Chem 272, 16010-7. Alternatively, green fluorescent protein may be used as a reporter (Phelan et al., 1998, Nat Biotechnol 16, 440-3).

Any of the domains or sequences or as set out above or identified as having translocational activity may be used to direct the agents (including biological effector molecules) into the cytoplasm or nucleus of a cell.

Nucleic Acid

A nucleic acid of use in the invention may comprise a viral or non-viral DNA or RNA vector, where non-viral vectors include, but are not limited to, plasmids, linear nucleic acid molecules, artificial chromosomes, condensed particles and episomal vectors. Expression of heterologous genes has been observed after injection of plasmid DNA into muscle (Wolff J. A. et al., 1990, Science, 247: 1465-1468; Carson D. A. et al., U.S. Pat. No. 5,580,859), thyroid (Sykes et al., 1994, Human Gene Ther., 5: 837-844), melanoma (Vile et al., 1993, Cancer Res., 53: 962-967), skin (Hengge et al., 1995, Nature Genet., 10: 161-166), liver (Hickman et al., 1994, Human Gene Therapy, 5: 1477-1483) and after exposure of airway epithelium (Meyer et al., 1995, Gene Therapy, 2: 450-460).

As used herein, the term “nucleic acid” is defined to encompass DNA and RNA or both synthetic and natural origin which DNA or RNA may contain modified or unmodified deoxy- or dideoxy- nucleotides or ribonucleotides or analogues thereof. The nucleic acid may exist as single- or double-stranded DNA or RNA, an RNA/DNA heteroduplex or an RNA/DNA copolymer, wherein the term “copolymer” refers to a single nucleic acid strand that comprises both ribonucleotides and deoxyribonucleotides.

The term “synthetic”, as used herein, is defined as that which is produced by in vitro chemical or enzymatic synthesis.

Therapeutic nucleic acid sequences useful according to the methods of the invention include those encoding receptors, enzymes, ligands, regulatory factors, and structural proteins. Therapeutic nucleic acid sequences also include sequences encoding nuclear proteins, cytoplasmic proteins, mitochondrial proteins, secreted proteins, plasmalemma-associated proteins, serum proteins, viral antigens, bacterial antigens, protozoal antigens and parasitic antigens. Therapeutic nucleic acid sequences useful according to the invention also include sequences encoding proteins, lipoproteins, glycoproteins, phosphoproteins and nucleic acids (e.g., RNAs such as ribozymes or antisense nucleic acids). Ribozymes of the hammerhead class are the smallest known, and lend themselves both to in vitro synthesis and delivery to cells (summarised by Sullivan, 1994, J. Invest. Dermatol., 103: 85S-98S; Usman et al., 1996, Curr. Opin. Struct. Biol., 6: 527-533). Proteins or polypeptides which can be expressed by nucleic acid molecules delivered according to the present invention include hormones, growth factors, neurotransmitters, enzymes, clotting factors, apolipoproteins, receptors, drugs, oncogenes, tumour antigens, tumour suppressers, structural proteins, viral antigens, parasitic antigens and bacterial antigens. The compounds which can be incorporated are only limited by the availability of the nucleic acid sequence encoding a given protein or polypeptide. One skilled in the art will readily recognise that as more proteins and polypeptides become identified, their corresponding genes can be cloned into the gene expression vector(s) of choice, administered to a tissue of a recipient patient or other vertebrate, and expressed in that tissue.

Delivery of Agents

The method of the present invention is useful for the delivery of agents to a selected site in a vertebrate body, whether an organ, part of an organ, cell, tissue type, or otherwise, in the presence or absence of specific targeting means. This is achieved, as set out above, by the selective disruption by ultrasound at the selected target site of electrosensitised red blood cells loaded with the agent of choice.

Agents useful for use in the present invention are set out above. Preferred agents include those useful for imaging of tissues in vivo or ex vivo. For example, imaging agents, such as antibodies which are specific for defined molecules, tissues or cells in an organism, may be used to image specific parts of the body by releasing them at a desired location using ultrasound. This allows imaging agents which are not completely specific for the desired target, and which might otherwise lead to more general imaging throughout the organism, to be used to image defined tissues or structures. For example, an antibody which is capable of imaging endothelial tissue may be used to image liver vasculature by releasing the antibody selectively in the liver by applying ultrasound thereto.

Kits

The invention also encompasses a number of kits. Some of the kits comprise partially or fully treated red blood cells. Other kits provide a red blood cell, an agent and packaging materials therefor together with instructions for carrying out the methods of the invention.

A kit designed for the easy delivery of an agent to a recipient vertebrate, whether in a research of clinical setting, is encompassed by the present invention. A kit takes one of several forms, as follows:

A kit for the delivery of an agent to a subject vertebrate comprises red blood cells and the agent and instructions for performing the method of the present invention. Alternatively, the red blood cells are supplied loaded with the agent for convenience of use by the purchaser. In the latter case, the cells are supplied in sensitised form, ready for rapid use or pre-sensitised and loaded but needing a final sensitisation step.

The cells of the kit are typically species-specific to the vertebrate of interest, such as a primate, including a human, canine, rodent, pig or other, as desired; in other words, the cells are of like species with the intended recipient. The cells of the kit are, additionally, specific to the blood type of the intended recipient organism, as needed. Optionally, the kit comprises one or more buffers for cell sensitisation, washing, re-suspension, dilution and/or administration to a vertebrate. Appropriate buffers are selected from the group that includes low ionic strength saline, physiological buffers such as PBS or Ringer's solution, cell culture medium and blood plasma or lymphatic fluid. The kit additionally comprises packaging materials (such as tubes, vials, bottles, or sealed bags or pouches) for each individual component and an outer packaging, such as a box, canister or cooler, which contains all of the components of the kit. The kit is shipped refrigerated. Optionally, non-cellular components are supplied at room temperature or frozen, as needed to maintain their activity during storage and shipping. They may be in liquid or dry (i.e., powder) form.

A second kit of the invention comprises an agent such as a biological effector molecule, instructions for performing the method of the present invention and, optionally a sensitising device and buffers therefor (e.g., saline or other physiological salt buffer, culture medium, plasma or lymphatic fluid). In addition, the kit contains appropriate packaging materials, as described above. The individual components may be supplied in liquid or dry (i.e., powder) form, and may be at room temperature, refrigerated or frozen as needed to maintain their activity during storage and shipping. Red blood cells for use with this kit may be obtained independently (for example, they may be harvested from the intended recipient vertebrate).

A preferred aspect of the invention is a kit comprising a red blood cell which is loaded with an agent, and packaging materials therefor.

Preferably, a kit as described above further comprises an apparatus for applying the sensitising procedure.

Preferably a kit of the invention further comprises polyethylene glycol. Preferably the kit further comprises a liquid selected from a buffer, diluent or other excipient. More preferably the liquid is selected from a saline buffer, a physiological buffer and plasma.

Another aspect of the invention is a physiological composition comprising a red blood cell delivery vector of the invention comprising a biological effector molecule admixed with a physiologically compatible buffer. As used herein, the term “physiologically compatible buffer” or “physiological buffer” is defined as a liquid composition which, when placed in contact with living cells, permits the cells to remain alive over a period of minutes, hours or days. As such, a physiological buffer is substantially isotonic with the cell, such that cell volume does not change more than 20% due to differences in internal and external ionic strength. Non-limiting examples of physiologically compatible buffers or physiological buffers include dilute saline, which may be buffered (e.g., Hanks' buffered saline or phosphate buffered saline), or other physiological salts (e.g., Ringer's solution), dilute glucose, sucrose or other sugar, dilute glycerol with- or without salts or sugars, cell culture media as are known in the art, serum and plasma.

Preferably, the red blood cell of the physiological composition is a human cell.

### EXAMPLES

### Example 1

### Loading of RBC with Oligonucleotides

In the following example three protocols for the loading and sensitisation of red blood cells (RBC/erythrocytes) are demonstrated and compared.

The first procedure demonstrates loading and sensitisation of red blood cells by exponential wave electric or square wave electric field pulse loading. Such electric pulses used for loading or sensitisation are abbreviated as ES. The second procedure consists of loading and sensitisation of red blood cells by a combination of electrosensitisation followed by hypoosmotic dialysis loading (HD, dialysis or osmotic loading). The combination is abbreviated as ES+HD. The third procedure consists of loading and sensitisation of red blood cells by a method comprising electrosensitisation (pre-sensitisation), followed by hypoosmotic dialysis, overnight rest and further treatment of the cells by electrosensitisation. This combination is abbreviated as ES+HD+ES.

In the first procedure, red blood cells are loaded with an oligonucleotide by a conventional electroporation procedure, as described in the prior art, using exponential wave electric pulses. Briefly, human blood was harvested by venipuncture and washed twice in PBS (phosphate buffered saline) by centrifugation. Cells were suspended in PBS containing 60 μg/ml of a random 30-mer FITC-labelled oligonucleotide to yield concentrations of 3.5×10⁸ cells/ml and 0.8 ml aliquots were dispensed into sterile electroporation cuvettes (0.4 cm electrode gap) and retained on ice for 10 min. Cells were then exposed to an electroporation strategy involving delivery of two electric pulses (field strength=3.625 kV/cm at a capacitance of 1 μF) using a BioRad Gene Pulser apparatus. Cells were immediately washed with PBS containing MgCl₂ (4 mM) (PBS/Mg) and retained at room temperature for 30 min in the PBS/Mg buffer to facilitate re-sealing. Cells were subsequently washed and suspended at a concentration of between 7 and 14×10⁸ cells/ml in PBS/Mg containing 10 mM glucose (PBS/Mg/glucose) for at least 1 hour.

The second procedure employed is essentially as described in our UK patent application 9917416.1, incorporated by reference. Briefly, 10 ml of peripheral venous blood is collected by venipuncture, into lithium heparin anticoagulant containing tubes, and mixed gently. The whole blood is then poured into a polypropylene tube and centrifuged at 300 g for 15 min at room temperature. The plasma and white blood cells (buffy coat) are removed.

1× phosphate buffered saline (PBS, made from Oxoid tablets code BR14a pH 7.3) is added and the cells centrifuged at 700 g for 5 min. The supernatant is removed and the pellet of remaining cells resuspended in ice cold 1×PBS. The spin/wash procedure is then repeated once, and cells are suspended in ice-cold PBS at 6×10⁸ cells/ml.

Cells are then electrosensitised by dispensing 800 l of the RBC into sterile electroporation cuvettes, and placed on ice. To electrosensitise the cells, they are exposed to an electric field at 3.625 kV/cm, 1 F (2 pulses), in the absence of payload. The RBCs are then removed, and pooled in polypropylene tubes.

Cells are centrifuged once at 700 g for 5 min at room temperature (RT). The cells may be diluted in PBS/MgCl₂ (4 mM). Cells are then re-suspended in PBS/MgCl₂, and centrifuged at 700 g for 5 min, twice. Finally, cells are re-suspended in PBS/MgCl₂, at approximately 7×10⁸ c/ml, and rested for 30 min at room temperature.

Cells are then loaded with oligonucleotide by hypoosmotic dialysis, according to a protocol adapted from Eichler et al., (1986) Clin. Pharmacol. Ther. 40:300-303. The following protocol is followed:

1 Buffers:

Stock potassium phosphate buffer:

5 mM K₂HPO₄ 3H₂O (FW 228.2 g) [symbol] 1.141 g/L

5 mM KH₂PO₄ (MW136.1 g) [symbol] 0.68 g/L

Stored at 4° C.

Mix as follows:

For a pH 7.4 K₂H/KH₂ phosphate buffer [symbol] approx. 6.1:3.9 parts

Mix the 2 stock solutions as and when required

Buffer #1 (isoosmotic PBS):

pH 7.4 K₂H/KH₂ phosphate buffer

150 mM NaCl [symbol] 8.76 g/L

Check and adjust pH (1M NaOH)

Buffer #2 (dialysis buffer):

pH 7.4 K₂H/KH₂ phosphate buffer

Check and adjust pH (1M NaOH)

Buffer #3 (resealing buffer)

pH 7.4 K₂H/KH₂ phosphate buffer

150 mM NaCl [symbol] 8.76 g/L

10 mM glucose [symbol] 1.8 g/L

Check and adjust pH (1M NaOH)

2 Spectrapor Dialysis Tubing:

1 The 3.5 kDa MW cut off tubing, 0.32 ml/cm, is used.

2 Preparation: heat at 80° C./30 min in 1 mM EDTA/2% sodium bicarbonate (Sigma). Rinse well, inside and outside, with ddH₂O.

3 Wash inside and outside with Buffer #1 4 Store submerged in a small amount of Buffer #1 if not used immediately.

3 RBC Preparation:

1 Electrosensitised, rested RBC are washed in PBS twice at 700 g for 5 min.

2 For the final wash, cells are washed in buffer #1

3 The cells are manipulated as a suspension of packed cells following removal of final wash supernatants after centrifugation.

4 Cell Volume in Tubing:

1 Protocol recommends 60% haematocrit (HCT). The suspension of packed cells is approximately 75% HCT and is diluted accordingly.

2 Mix cells with the oligonucleotide and buffer #1, to give required final oligonucleotide concentration and volume.

5 Dialysis:

1 The tubing is clipped to ensure that the surface area remains constant for the volume of cells.

2 Dialyse RBC (packed cell volume in buffer #1) against buffer #2 for 90 min at 4° C.

3 Place membranes in 100-200 ml buffer #2, (ensure that the membrane is immersed) in glass beaker with magnetic flea.

4 Place this beaker within another beaker, which contains ice, on the magnetic stirrer, cover with silver foil.

6 Warm up an aliquot of buffer #3 to 37° C.

7 Remove dialysis buffer, replace with the warm resealing buffer #3.

8 Place beaker with dialysis tubing and buffer #3 into a larger beaker anchored by water at 37° C., cover and leave for 15 min.

9 Harvest cells into 12 ml polypropylene tubes.

10 Wash ×3 in ice cold resealing buffer #3 at 300 g, 10 min 4° C.

11 Wash ×1 in PBS/Mg/glucose and spin at 700 g, 5 min 4° C.

12 Count cells and resuspend at 7×10⁸ c/ml, in PBS/Mg/glucose.

13 Store at 4° C. overnight.

In the present example, dialysis is performed in the presence of 10 μg of oligonucleotide per ml of cells. Cells are suspended at 7×10⁸ cells/ml.

In the third procedure, cells are prepared as described for the second procedure, but exposed to an additional electrosensitisation step after loading by dialysis, according to the following protocol.

1 Following overnight storage, wash RBC once in PBS 700 g, 5 min 4° C.

2 Count cells and resuspend at 6×10⁸ c/ml, in ice cold PBS.

3 Dispense 800 l of the RBC into sterile electroporation cuvettes (0.4 cm gap).

4 Place on ice.

5 To electrosensitise: double pulse at 3.625 kV/cm, 1 F.

6 Harvest the RBC, pool in a polypropylene tube.

7 Centrifuge once at 700 g for 5 min room temperature (RT). The cells may be diluted in PBS/MgCl₂(4 mM).

8 Resuspend in PBS/MgCl₂, centrifuge at 700 g for 5 min.

9 Repeat step 6

10 Resuspend in PBS/MgCl₂, at approximately 7×10⁸ c/ml.

11 Rest the cells for 30 min at RT.

12 Centrifuge once at 700 g for 5 min room temperature (RT). The cells may be diluted in PBS/MgCl₂/glucose.

13 Resuspend the cells in PBS/MgCl₂/glucose, centrifuge at 700 g for 5 min.

14 Repeat step 13.

15 Resuspend cells in PBS/MgCl₂/glucose at 7×10⁸ c/ml.

16 Rest the cells in PBS/MgCl₂/glucose for 60 min.

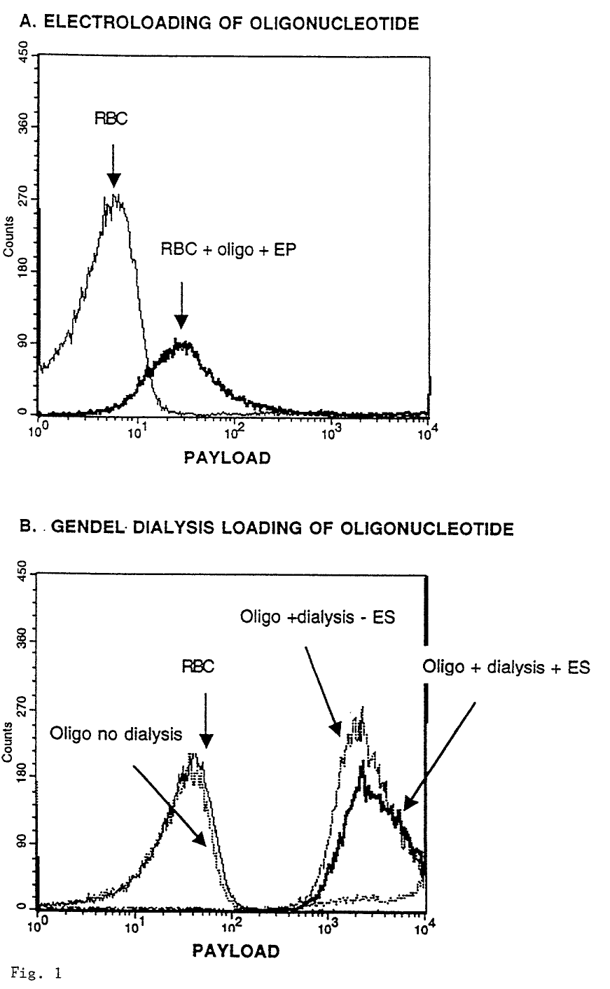

Cells prepared according to all three procedures were analysed to determine cell loading levels and subjected to ultrasound disruption. The results are shown in FIG. 1.

For the electroporated cells, shown in FIG. 1A, the oligonucleotide (oligo) did not bind non-specifically to RBC as the mean fluorescence intensity (MFI) was 1. The MFI is defined as the ratio of fluorescence associated with loaded cells divided by the fluorescence associated with non-specific binding. The electroloaded cells have an increase in fluorescence, with an MFI of 5.6.

Ultrasound sensitivity was measured at 0.75 W/cm², 3 MHz, 30 sec. 0% of control cells lysed, compared with 20% of the electroporated cells.

For cells loaded by electrosensitisation followed by hypoosmotic dialysis (ES+HD), non-specific binding is negligible (MFI=1). Loading by ES+HD results in a marked increase in fluorescence with an MFI of 80. Incorporation of the second electrosensitisation step (ES+HD+ES) has little effect on the level of fluorescence with an MFI of 93 indicating retention of the payload during the procedure.

Ultrasound sensitivity is measured at 3 W/cm², 1 MHz, 35 sec in a TMM (tissue mimicking medium). Cells subjected to only a single electrosensitisation and dialysis procedure (ES+HD) show 28% lysis, whilst cells subjected to the additional electrosensitisation step (ES+HD+ES) show 89% lysis.

### Example 2

### Loading of RBC with Antibodies

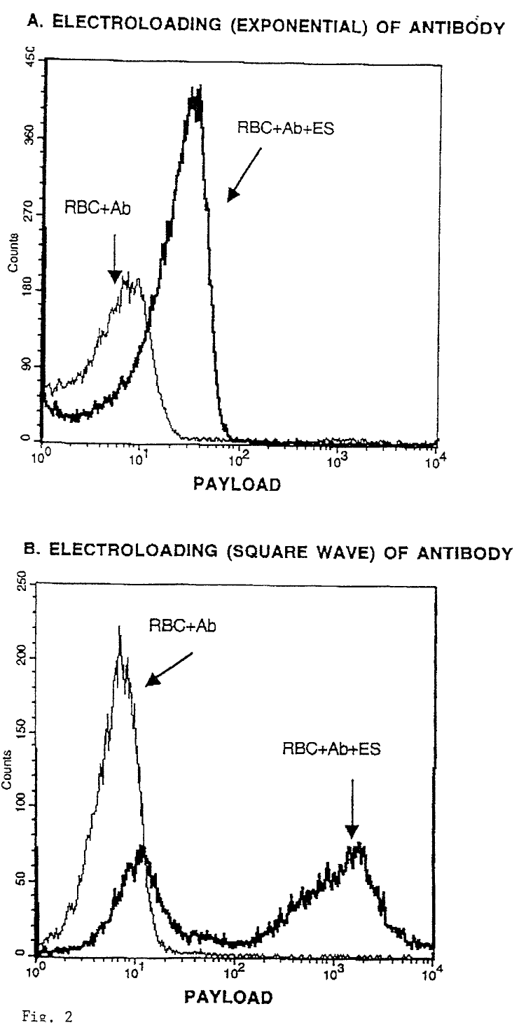

For comparative purposes, antibodies are loaded into RBC by electroporation. The antibody used is a FITC-conjugated anti-vWF antibody (Sigma). The results are shown in FIG. 2.

In a first procedure (FIG. 2A), cells are loaded as described in Example 1 by exposing 3.5×10⁸ cells/ml to 3 pulses of an exponential wave electric field in the presence of antibody at 0.25 mg/ml. An MFI of 3.98 is observed, with 100% ultrasound sensitivity. However, in this procedure the cell recovery is low, approximately 11%.

In a second procedure (FIG. 2B), 10 pulses of a square wave electric field are used, in the presence of 0.5 mg/ml antibody (the conditions are optimised for each protocol). Two peaks are seen in the cell population after loading, with an MFI of 1.73 and 184.8. 40% of the cells are recovered, of which 97% are ultrasound sensitive when exposed to ultrasound at an energy of 1.25 W/cm using a 3 MHz probe for 30 s. 0.005 pg antibody per cell was recovered.

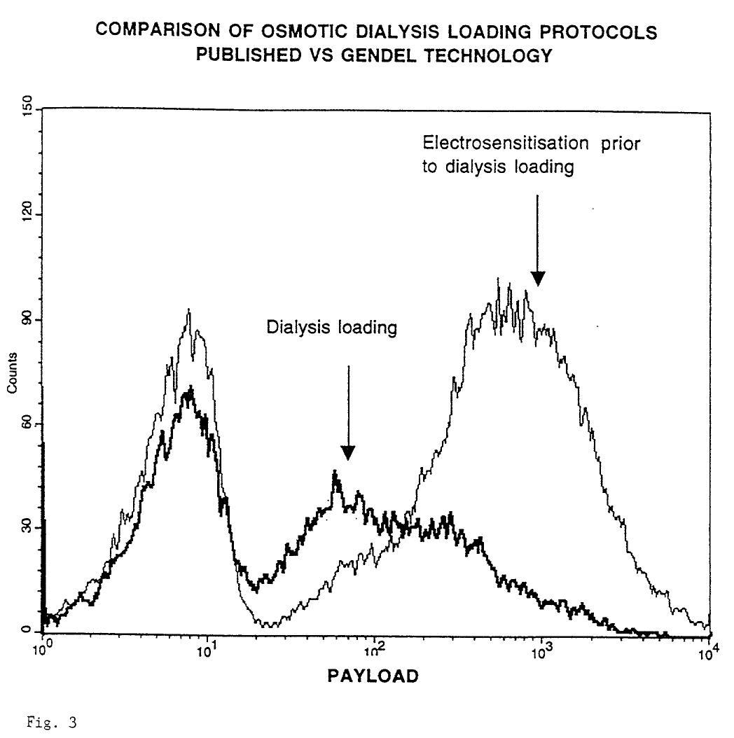

FIG. 3 shows the results of hypoosmotic dialysis loading of antibody according to the procedure of Eichler et al. and of the present invention (see Example 1). The relative MFI is 15.2 in the absence of electrosensitisation (HD loading alone) compared to an MFI of 61.7 in the presence of electrosensitisation (ES+HD). This demonstrates a dramatic increase in loading.

In both dialysis protocols, 80 to 90% of the cells are recovered. Ultrasound sensitivity is about 30% in the absence of a second sensitisation step (ES+HD). Following the second sensitisation step (ES+HD+ES) there is no apparent leakage/loss of the payload and about 90-100% of the cells are ultrasound sensitive (see table in Example 6). 0.22 pg antibody/cell was recovered.

### Example 3

### Resealing Buffers

An alternative resealing buffer is tested in order to assess any impact on the performance of the method according to the invention. The buffer of Bax et al., (1999) Clinical Science 96:171-178, is compared to the buffer adapted from Eichler et al. as used in Example 1 (ES+HD).

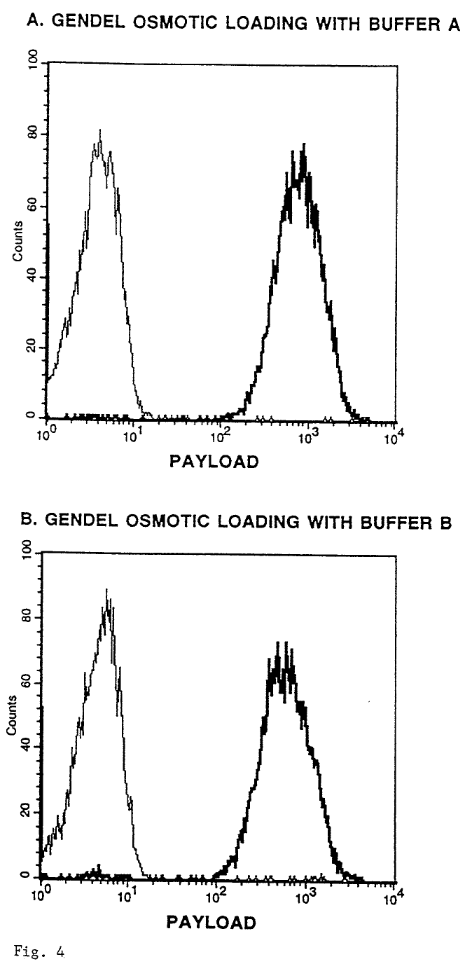

As can be seen from FIG. 4, the performance of the two buffers is almost identical. In both cases, cells were loaded with FITC conjugated anti-vWF antibody as described in Example 2, and subjected to a second electrosensitisation procedure in accordance with the present invention.

The mean fluorescence intensities observed for the Eichler and Bax buffers are 186 and 126 respectively. Observed cell losses are 27% and 16%.

Ultrasound sensitivities are measured in a TMM, at 3 W/cm², for 35 seconds; cell lysis of 68% and 72% is observed, respectively.

### Example 4

### Ultrasound-mediated Release of Antibody Payload in a Perfused Rat Kidney System

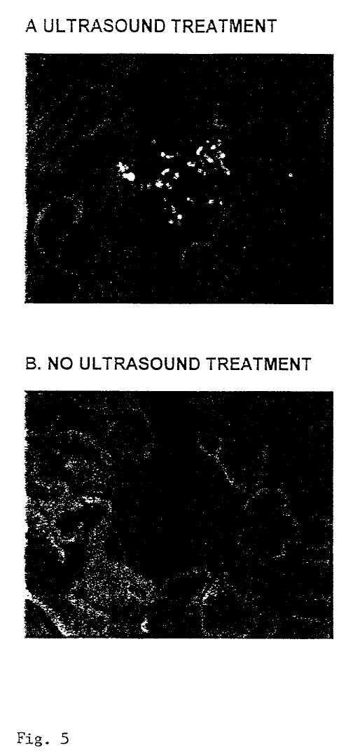

The release of FITC-conjugated antibody by ultrasound in PBS-perfused kidney tissue is shown in FIG. 5. RBC are loaded with FITC-anti-vWF antibody as described in Example 2 above, in accordance with the present invention, and administered to PBS-perfused kidneys, according to the following protocol:

1. Perfuse the rat through the heart with PBS/EDTA until the kidney is clear of blood

2. Remove the dorsal aorta from the heart and insert a gavage needle into the vessel. Tie the needle to the dorsal aorta using suture.

3. Close the dorsal aorta and posterior vena cava just after the junction leading to the kidney.

4. Close the left adrenal artery and vein and both anterior mesenteric and coeliac arteries

5. Close the ureter and the left iliolumbar artery and vein.

6. Create an exit point by inserting a gavage needle into the vena cava just before the liver. Tie the needle using suture.

7. Flush with 10 ml PBS/4 mM Mg/10 mM glucose and check for any leakage.

8. Block the exit point by inserting 2 ml syringe into the gavage needle.

9. Load 1 ml of 7×10⁸ cells/ml through the dorsal aorta into the kidney.

10. Treat with U/S using 1 MHz probe.

11. Incubate the treated kidney for one hour

12. Remove the 2 ml syringe and flush through with 2 ml PBS/Mg/glucose

13. Collect the flush through for cell counting and ELISA

14. Flush with 50 ml of PBS/EDTA

15. Flush with 20 ml of 4% neutral buffered formalin (NBF)

16. Remove the U/S-treated kidney and cut it into two half's and fix in NBF

17. Prepare tissue sections (12 m)and stain using Vectastain ABC kit (Vecta Labs) as outlined in the manufacturer's instructions.

Preparation of RBC: dialysed and electrosensitised (ES+HD+ES), antibody loaded erythrocytes:

Rat 1 No ultrasound treatment

Rat 2 Ultrasound treatment at 3 W/cm² for 40 seconds.

Kidney endothelial cells in glomeruli are labelled by the FITC conjugated anti-vWF antibody after ultrasound treatment to release the antibody, as shown in FIG. 5A. In the absence of ultrasound treatment, no staining is observed (FIG. 5B).

### Example 5

### Stability of Loaded Cells

RBC are loaded by dialysis according to the present invention (ES+HD+ES), as described in Examples 1 and 2, with FITC-conjugated antibody. Following the second sensitisation, cells are stored at 7×10⁸ cells/ml in SAGM buffer (Blood transfusion service buffer, obtainable from Baxter Health Care). Cells are stored with maximal exclusion of air at 4° C. Maintenance of ultrasound sensitivity, cell numbers and payload are assessed over a period of 35 days.

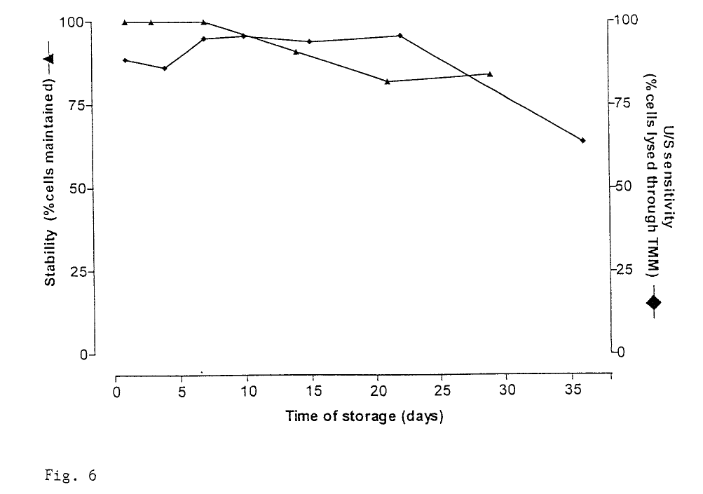

FIG. 6 shows the levels of cell numbers and ultrasound sensitivity in cells on storage. Ultrasound sensitivity, measured at 3 W/cm², 35 sec, in a TMM, is maintained at or above the starting level of 90% for 25 days, and falls to about 65% after 35 days. Cell numbers are stable over a 30 day period.

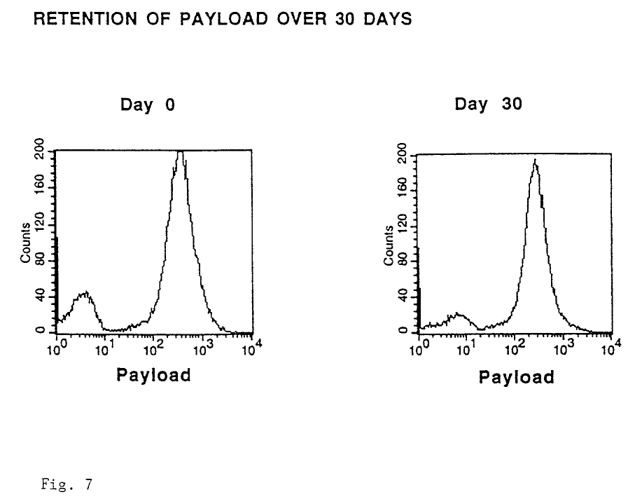

FIG. 7 shows the retention of payload over 30 days under identical conditions to the above. No loss of payload is observed.

### Example 6

### Comparison of Different Sequences of Sensitisation and Osmotic Loading Steps

The hypoosmotic dialysis loading protocol described in Example 1 is performed in two different configurations to determine the effect on loading efficiency and susceptibility to ultrasound mediated lysis when the loading step is performed before the second sensitisation step, as in Example 1, and vice versa.

Electrosensitisation steps and dialysis were carried out as described in Example 1, except that the electrosensitisation steps are carried out twice. Ultrasound sensitivity is determined in a TMM as described in Example 1.

TABLE 1

| | | % U/S | |
|---|---|---|---|
| | % U/S | mediated lysis | Mean |
| | mediated lysis | following rest | fluorescence |
| Sample | day of load | overnight | intensity |
| RBC | 0 | 5 | — |
| ES + dialysis | 0 | 0 | 66 |
| (ES + HD) | | | |
| ES + dialysis + ES | 0 | 90-100% | 61.7 |
| (ES + HD + ES) | | | |
| ES + ES + dialysis | 84 | 95 | 79 |
| (ES + ES + HD) | | | |

The results shown above in Table 1, similar to those obtained in Example 1, indicate that the sequence of the two sensitisations is not critical to obtaining improved sensitivity.

### Example 7

### Release of Payload from Loaded and Sensitised Vehicle in a Tissue Mimicking System (TMM)

It has been demonstrated that enhanced loading of cells may be achieved by exposing to electric fields in combination with hypoosmotic dialysis loading modalities. In the studies presented in the examples described here, cells were loaded with antibody, enzyme and oligonucleotide by firstly exposing the cells to pre-sensitising electric pulses and subsequently carrying out hypoosmotic loading. The cells were then electrosensitised by exposing to electric pulses. This protocol is defined by ES+HD+ES in the previous examples (see Example 1). In the experiments described here the objective was to demonstrate enhanced loading to confirm earlier examples and to demonstrate ultrasound mediated release of the relevant payload using a tissue mimicking system. In these experiments the target was placed at a distance of 1.3 cm from the emitting surface of the ultrasound head and the intervening space was filled with a tissue mimicking material (TMM) which attenuates ultrasound in the same manner as a soft tissue. The TMM chosen for this work has been described by Madsen et al. (1998, Ultrasound Med. & Biol., 24, 535-542) and following preparation, care was taken to ensure that the material had a density of 1.03 g/ml.

I. Ultrasound-mediated Release of Antibody from the Vehicle

Antibody was loaded into the erythrocytes and sensitisation were carried out using the procedure denoted by ES+HD+ES as described for Example 1. Antibody-loaded sensitised cells were then exposed to ultrasound at a distance of 1.3 cm from the emitting surface of the ultrasound head. The intervening space was filled with the TMM as described above and 0.1 ml aliquots of 7×10⁸ cells/ml were exposed to ultrasound. In these studies a sheep anti-human von Willebrand factor antibody was employed as the payload in these studies. The amount of antibody in cells and released following treatment with ultrasound was quantified using an ELISA system.

I. Results

In the loading and sensitisation protocol, cells were loaded at a concentration of 1.1 mg of antibody per ml of packed cell volume (PCV). 0.1 ml aliquots of cells at 7×10⁸ cells/ml were exposed to ultrasound at intensities shown in FIG. 7 and samples were analysed for cell lysis by direct counting. In addition, the amount of antibody released following treatment with ultrasound was determined by ELISA analysis of cell supernatants harvested following centrifugation. The results obtained are shown in FIG. 7 and they demonstrate that cells were preferentially lysed at ultrasound power densities greater than 2 W/cm². Control cells exhibited little or no effect when treated with ultrasound at these power densities. In addition, at and above 2 W/cm² antibody payload was detected in supernatants harvested following ultrasound treatment. In addition, when the total amount of antibody released from the cells using ultrasound was compared with that released following hypotonic lysis in 0.01% (v/v) Triton X100 it was found that 77% of the total antibody was released in the former. The remainder could be found in debris that was recovered by centrifugation following ultrasound treatment.

The results demonstrated that treatment of cells with the ES+HD+ES protocol results in sensitivity of that loaded population to ultrasound. Ultrasound-mediated payload release could be achieved using low intensity ultrasound and using conditions which had little or no effect on normal erythrocytes. The results also demonstrate ultrasound-mediated release of payload at a depth of 1.3 cm and thereby demonstrating one of the major advantages associated with the use of ultrasound as the releasing stimulus of penetration to depth in tissues. Since all of the antibody incorporated into the ultrasound treatment experiments could be recovered as shown using an ELISA based on payload functionality, this suggested that the ultrasound had no detrimental effect on the functionality.

II. Ultrasound-mediated Release of Enzyme (β-galactosidase) from the Vehicle

Cells were harvested, pre-sensitised by exposure to electric pulses and loaded with β-galactosidase (from Escherichia coli, Sigma) as described above for antibody loading. Cells were subsequently exposed to sensitising electric pulsing and exposed to ultrasound at a concentration of 7×10⁸ cells/ml in the TMM system as described above for the antibody-loaded vehicle. Lysates obtained following exposure of the loaded and sensitised vehicle to ultrasound were assayed for β-galactosidase activity at 37° C. using the calorimetric substrate p-nitrophenyl-β-D-galactoside (5 mM in 50 mM phosphate buffer, pH 7.0). The concentration of p-nitrophenol was determined spectrophotometrically at 450 nm and activity was expressed as μmoles of p-nitrophenol produced per minute per ml of sample. Release of enzyme in samples harvested following treatment with ultrasound was expressed as a percentage relative to the amount of enzyme contained in the cells prior to treatment. The latter was determined by measuring the amount of enzyme released from the cells following lysis by freeze-thaw in 5 mM phosphate buffer, pH 7.2.

II. Results

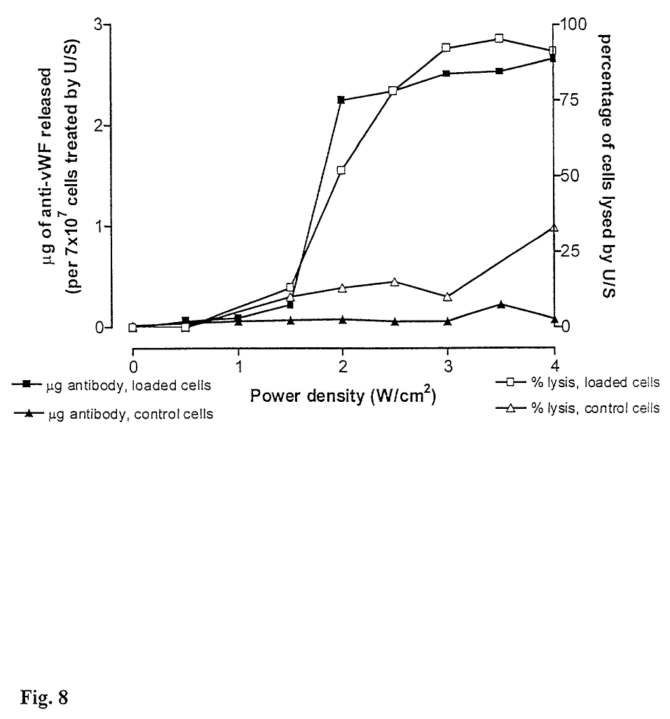

In these experiments loaded cells contained approximately 1 mg of enzyme per ml of packed cell volume. The results obtained following treatment of these preparations with ultrasound are shown in FIG. 8. Samples were treated at the indicated power densities as shown and samples were analysed for cell lysis by cell counting. Lysis increased with increasing power density up to a maximum at about 3 W/cm². Exposure of control normal cells to similar ultrasound conditions had little or no effect on cell lysis and this was confirmed by the absence of haemoglobin in supernatants following removal of cells by centrifugation. When supernatants were harvested by centrifugation, following exposure of the sensitised and loaded cells to ultrasound and analysed for enzyme content, it was found that increasing amounts of enzyme were released with increasing power density up to a maximum at 3 W/cm².

The results demonstrated cells loaded using the ES+HD+ES protocol are sensitive to ultrasound and the enzyme payload may be released from the vehicle following exposure to low intensity ultrasound. Ultrasound-mediated release of the payload is achieved at 1.3 cm from the emitting ultrasound head, indicating that the use of ultrasound for this purpose offers the advantage of penetration to depth in tissues. In addition, since 100% recovery of the enzyme released was achieved (between 2.5-3 W/cm²), the ultrasound stimulus resulting in release of enzyme had no detrimental effect on the functionality of the released payload.

III. Ultrasound-mediated Release of Oligonucleotide from Vehicle

Cells were harvested and pre-sensitised by exposure to electric pulses as described above. Cells were then loaded using the hypoosmotic dialysis procedure described above for antibody loading and a 300 μg quantity of oligonucleotide (tamara labelled random 30-mer supplied by Oswel, UK) was mixed with 250 μl of packed cells. Samples were then subjected to electrosensitisation electric pulses and subsequently suspended in PBS/MgCl₂/glucose at a concentration of 7×10⁸ cells/ml. Samples were exposed to ultrasound using the TMM system described above for antibody and enzyme release and the amount of oligonucleotide released was determined using a spectrofluorimeter (Shimadzu) with excitation set at 540 nm and emission set at 590 nm. A standard curve was constructed for quantitative determinations and extraction efficiencies were taken into account.

III. Results

In these experiments the maximum amount of oligonucleotide loaded was approximately 300 μg of oligonucleotide per ml of packed cell volume.

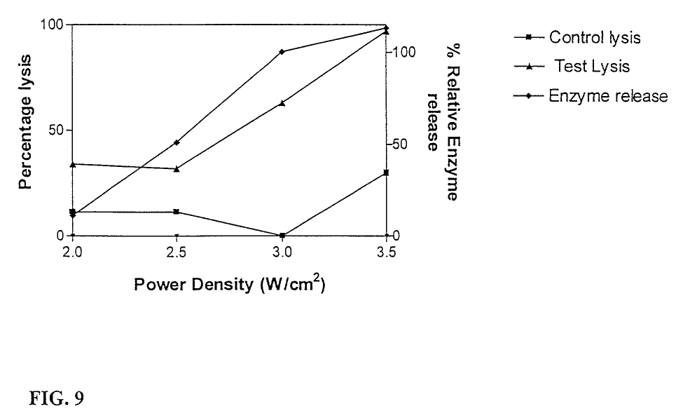

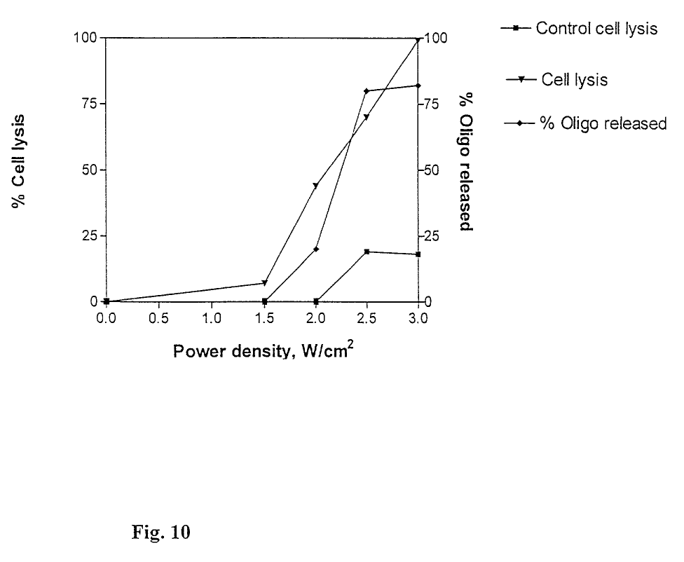

The results obtained following treatment of these loaded preparations with ultrasound are shown in FIG. 9. As with the above two examples, cell lysis of the sensitised and loaded preparation occurs between 2 and 3 W/cm². Under these ultrasound conditions there is little or no effect on control erythroyctes. In addition oligonucleotide begins to appear in harvested supernatants between 2 and 3 W/cm² demonstrating ultrasound-mediated release of oligonucleotide payload from the vehicle.

### EXAMPLE 8

### Enhanced Loading of Erythrocytes by Replacing Initial ES Step with a Sonoporative Treatment

The results above demonstrate that cells loaded using the ES+HD+ES protocol are more efficiently loaded than using ES or HD alone. These results also demonstrate release of the payload from the vehicle.

In many of the previous examples sensitisation and loading was achieved by pre-sensitising the cell using electrosensitisation and subsequently processing through hypoosmotic loading protocols and a further exposure to electric pulses (ES+HD+ES). The resulting preparations were efficiently loaded with the relevant payload and the preparations also exhibited sensitivity to ultrasound. Since it was felt that the initial pre-sensitising event, which is reported to create transient poration of the membrane, contributed positively to loading by hypoosmotic loading it was of interest to determine whether or not other porative methods might contribute in a similar manner. Sonoporation represents an alternative technique known to create transient membrane poration. This involves the use of ultrasound and has been reported to aid in creating non-destructive and transient poration of biological membranes (Miller et al, 1998, Ultrasonics 36, 947-952). It was therefore of interest to determine whether or not exposure of erythroyctes to ultrasound prior to hypoosmotic loading would contribute positively to loading of those cells and if so, could those cells be rendered sensitive to ultrasound by subsequently exposing them to sensitising electric pulses.

To the above ends, human erythroyctes were harvested and loaded with fluorescein-labelled anti-rat IgG using either the original electrosensitisation (pre-sensitisation)—hypoosmotic dialysis—electrosensitisation protocol (ES+HD+ES), hypoosmotic dialysis alone (HD) and a sonoporation—hypoosmotic dialysis—electrosensitisation (SP+HD+ES) protocol. The former two were performed as described above and the latter consisted of the original ES-HD-ES protocol except that the first ES step was replaced by a sonoporative step.

This involved suspending washed erythrocytes in PBS at a concentration of 7×10⁸ cells/ml. 1.5 ml aliquots were then dispensed into individual wells to a 24-well tissue culture plate. Cells were subjected to ultrasound treatment at 2.5 W/cm² for 5 min. using a 1 MHz ultrasound head. After treatment cells were washed by centrifugation and suspended in PBS/MgCl₂. Cells were then processes as for the ES+HD+ES protocol and loading of antibody was assessed using flow cytometry. In addition, the sensitivity of those cells was determined by exposing the cells to ultrasound at 3 W/cm² using the TMM system.

Results 8

When cells were treated with the ES+HD+ES protocol and subsequently exposed to ultrasound at 3 W/cm² using the TMM over 90% lysis was obtained and this is in agreement with previous results. When cells were treated with the SP+HD+ES protocol and exposed to ultrasound in the above manner 30% lysis occurred. The results demonstrate that the cells have been sensitised using the alternative protocol although not to the same degree as that achieved with the original protocol involving the use of the preliminary pre-sensitising electrosensitisation step.

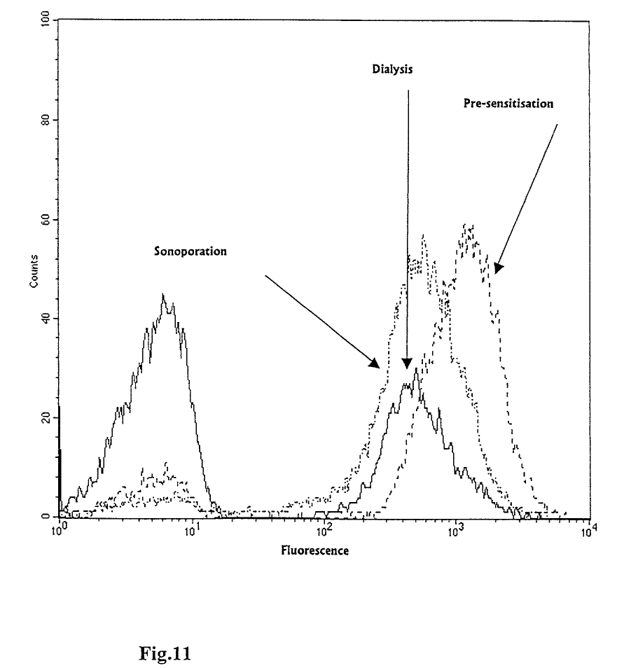

When loading of the cells was examined using flow cytometry it was found that the loading with HD alone resulted in two peaks of fluorescent cells as shown in FIG. 11. This indicated loading of the cells was inefficient since over half of the population of cells remained unloaded or minimally loaded. When cells were loaded using the ES+HD+ES protocol, a major peak shifted to the right was detected and this indicated that almost all (88%) of the cells in the population were maximally loaded (FIG. 11). When the protocol employing a sonoporative pre-sensitisation step prior to hypotonic dialysis was analysed on flow cytometry it was found that again, most of the cells resided in a peak shifted well to the right in FIG. 11. This again indicated that almost all of the cells (93%) were loaded with fluorescent antibody. However since the shift to the right in this peak was not as great as that found in the sample treated with the ES+HD+ES protocol, the amount of fluorescent antibody associated with the cells in this peak is not as great and this is indicated by the mean fluorescent intensities listed in the Table 2 below. It should also be noted that yields using the sonoporative method were not as high as those from the alternative two methods. The ultrasound-mediated protocol provides advantage over hypotonic dialysis in terms of loading the full population of cells. Although the sonoporative protocol is not as efficient in terms of payload incorporated it does provide an alternative means of enhancing loading achieved with electrosensitisation (pre-sensitisation) and hypoosmotic dialysis alone.

When cells were loaded with the SP+HD protocol they did not exhibit sensitivity to ultrasound. However, when subsequent exposure to electric pulses was carried out i.e. SP+HD+ES, 30% lysis of the population (7×10⁸ cells/ml) was observed when treated using the TMM system described above.

TABLE 2

| | % of cells in | |
|---|---|---|
| Treatment | Second Peak | MFI of Second Peak |
| HD (95) | 40 | 32 |
| ES-HD-ES (88) | 88 | 39 |
| SP-HD-ES (60) | 90 | 27 |

MFI = mean flourescence intensity which is = flourescent intensity of sample/flourescent intensity exhibited as a result of non-specific binding
Values in parentheses represent the yields of cells obtained from each protocol

### Example 9

### Ultrasound-mediated Release of Payload from the Loaded, Sensitised Vehicle in a Circulating System at 37° C. and at High Hematocrit (HCT.)

In the above studies it is shown that human erythroyctes can be loaded at high efficiency, sensitised to low intensity ultrasound and ultrasound-mediated disruption and/or payload release can be achieved in vitro and in an ex vivo perfused rat kidney system. In these systems disruption and/or payload release is demonstrated at 7×10⁸ cells/ml which is approximately equivalent to a 5% HCT. In addition, those studies are performed at room temperature. It is of interest to demonstrate that sensitivity in terms of payload release can be retained at 37° C. and at higher HCT. It is also of interest to determine whether or not this occurs while the target cells are moving through a circulation system in much the same as those circulating in vivo.

To these ends human erythrocytes are harvested and loaded with anti-von Willebrand factor antibody as described for Example 7. Following sensitisation cells re mixed together with normal washed human cells in the proportions of one part 7×10⁸ cells/ml and four parts 4×10⁹ cells/ml. The mixture is introduced into a circulating system consisting of a cylindrical reservoir filled with PBS and maintained at 37° C. by circulation. The bottom of the cylinder consists of a light polyethylene sheet through which ultrasound is delivered. The blood is circulated through C-flex tubing (internal diameter 4 mm) which passes through the thermostated buffer and the target area of the C-flex tubing is positioned at a distance of 1.3 cm from the ultrasound-emitting head. Blood is circulated through the system at a rate of 14.5 ml/min. During exposure to ultrasound (5 W/cm² at 1 MHz for indicated times), samples are harvested from the system and supernatants are harvested by centrifugation. These are then assayed for antibody using an ELISA assay as described above. The control in these experiments consists of loaded and sensitised cells circulated through the system in the absence of ultrasound. It is also important to note that circulation of normal cells through the system while ultrasound is being delivered results in no apparent damage as determined by the lack of hemoglobin in supernatants following treatment.

Results 9

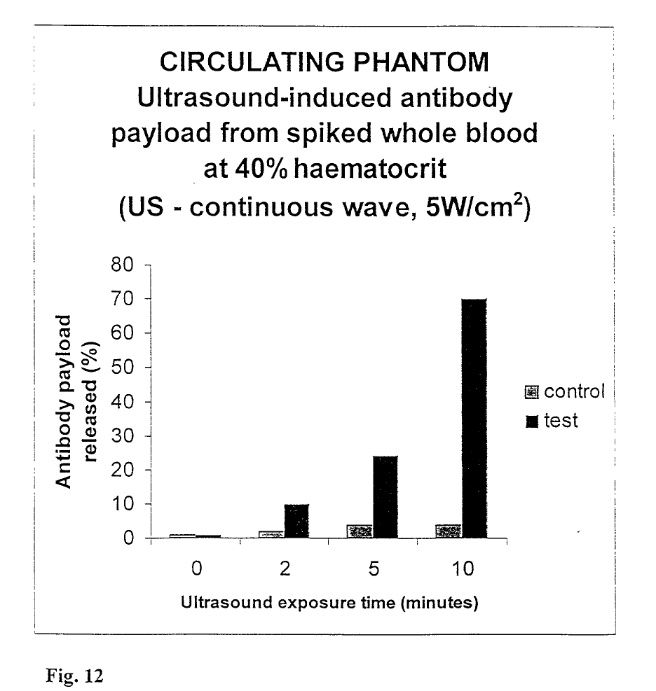

The results are shown in FIG. 12 and they demonstrate that detectable quantities of antibody are released from the vehicle between 2 and 5 minutes treatment with ultrasound. Little or no antibody can be detected in control samples which consist of the loaded and sensitised cells circulated through the system in the absence of ultrasound. The results demonstrate that the ultrasound-sensitisation phenomenon is intact at 37° C. and ultrasound-mediated release of payload is achieved at high hematocrit (HCT., i.e., 40%) and in a mobile target system.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

We claim:

1. A method of producing a red blood cell comprising an agent comprising:
(a) providing said red blood cell;
(b) pre-sensitising said red blood cell; and,
(c) loading said red blood cell with said agent.

2. A method according to claim 1 said step (c) comprising leading a first and said red blood cell with a second agent.

3. A method according to claim 1, further comprising the step of electrosensitising the cell.

4. A method for selectively releasing an agent from a red blood cell comprising the steps of:
(a) pre-sensitising a red blood cell;
(b) loading said red blood cell with an agent;
(c) electrosensitising said red bood cell; and
(d) effectuating substantial release of said agent from said sensitised red blood cell by applying ultrasound. at a frequency and energy sufficient to cause disruption of unsensitised red blood cells,

5. A method for delivering an agent in a vertebrate comprising,
(a) pre-sensitising a red blood cell;
(b) loading said red blood cell with an agent;
(c) electrosensitising said red blood cell;
(d) introducing said red blood cell into a vertebrate; and
(d) releasing said agent from said sensitised cell by ultrasound.

6. A method according to claim 5, wherein said red blood cell of step (w) is immunocompatibile with said vertebrate.

7. A method according to claim 5, in which the red blood cell is PEGylated prior to being introduced into the vertebrate.

8. A method according to claim 5 in which the vertebrate is a mammal.

9. A method according to claim 1 or claim 5 wherein one or both of said pre-sensitising or electrosensitising steps is performed in vitro or ex-vivo.

10. A method according to claim 1 or claim 5, wherein said pre-sensitising step comprises applying an electric field to said red blood cell.

11. A method according to claims 1 or claim 5, wherein said pre-sensitising step further comprises applying ultrasound to the red blood cell.

12. A method according to claim 1 or claim 5, wherein said loading step comprises hypotonic dialysis.

13. A method according to claim 3, wherein said electrosensitizing step comprises applying an electric field to said red blood cell.

14. A method according to claim 13, wherein said electric field applied to said red blood cell ranges from 0.1 kV/cm to 10 kV/cm.

15. A method according to claim 13, wherein said electric field is applied to said red blood cell 1 microsecond to 100 milliseconds.

16. A method according to claim 3, wherein said electrosensitisation step is performed after said loading step.

17. A method according to claim 3, wherein said electrosensitisation step is performed before said loading step.

18. A method according to claim 4, wherein said ultrasound is selected from the group consisting of diagnostic ultrasound, therapeutic ultrasound and a combination of diagnostic and therapeutic ultrasound.

19. A method according to claim 4 wherein the applied ultrasound energy source is at a power level from about 0.05 W/cm² to about 100 W/cm².

20. A red blood cell composition comprising a plurality of pre-sensitized red blood cells.

21. The red blood cell composition according to claim 20, wherein said red blood cell is pre-sensitized to permit loading of an agent.

22. A red blood cell composition according to claim 20 comprising a plurality of pre-sensitized electro sensitized red blood cells.

23. A red blood cell composition according to claim 20, wherein said red blood cells are immunocompatible in a vertebrate.

24. A red blood cell composition according to claim 20 wherein said agent is selected from a group consisting of: a protein, a polypeptide, a peptide, a nucleic acid, a peptide nucleic acid (PNA), a virus, a nucleotide, a ribonucleotide, a deoxyribonucleotide, a heteroduplex, a nanoparticle, an amino acid, a steroid, a proteoglycan, a lipid, a fatty acid, an oligosaccharide, a glycoprotein, and a carbohydrate.

25. A red blood cell composition according to claim 24 wherein said agent further comprises an imaging agent.

26. A red blood cell composition obtainable by a method comprising:
(a) presensitising a red blood cell;
(b) loading the cell with an agent; and
(c) electrosensitising the cell.

27. A kit comprising a red blood cell composition according to claim 20, and packaging materials therefor.

28. A kit comprising a pre-sensitised red blood cell, an agent, and packaging materials therefor.

29. A kit according to claim 27 or 28, said kit further comprising a liquid selected from the group consisting of: a buffer, diluent, an excipient, a saline buffer, a physiological buffer, serum, and plasma.

30. A pharmaceutical composition comprising a red blood cell composition made by a process comprising:
(a) providing a red blood cell;
(b) pre-sensitizing said red blood cell;
(c) loading said red blood cell with an agent; and
(d) electrosensitizing said red blood cell.

31. The composition of claim 31 wherein said red blood cell composition further comprises a red blood cell is immunocompatible in a vertebrate.

32. The composition of claim 31 wherein said red blood cell comprises PEG.

33. A device for producing a red blood cell delivery composition, comprising:
(a) one or more flow cells and electrosensitisation means;
(b) one or more dialysis systems;
in which the flow cell is linked to the dialysis system by connecting means capable of allowing transfer of red blood cells from the flow cell to the dialysis system.

34. A device as claimed in claim 33 wherein said device for sensitizing said red blood cell emits an electric field.

35. A device as claimed in claim 33 wherein said device for sensitizing said red blood cell emits ultrasound.
